(19) United States
(12) Patent Application Publication
(10) Pub. No.: US 2025/0082923 A1
(43) Pub. Date: Mar. 13, 2025

(54) INTRALUMINAL MODULAR POWERED MEDICAL SYSTEM

(71) Applicant: Puzzle Medical Devices Inc., Montreal (CA)

(72) Inventors: Gabriel GEORGES, Sherbrooke (CA); François TRUDEAU, Quebec (CA); Yves-Antoine CRÊTE, Montreal (CA)

(21) Appl. No.: 18/892,977

(22) Filed: Sep. 23, 2024

Related U.S. Application Data
(63) Continuation of application No. PCT/CA2023/050378, filed on Mar. 22, 2023.
(60) Provisional application No. 63/322,691, filed on Mar. 23, 2022.

(51) Int. Cl. A61M 60/878; A61M 60/178; A61M 60/216; A61M 60/865; A61M 60/873

(52) U.S. Cl. CPC A61M 60/878; A61M 60/178; A61M 60/216; A61M 60/865; A61M 60/873

(57) ABSTRACT

An intraluminal modular powered medical system includes: an intraluminal dock including at least one dock electrical connector configured to receive power from a power source; and an intraluminal medical device including at least one device electrical connector configured to intraluminally electrically connect to the at least one dock electrical connector for receiving power therefrom. The intraluminal dock and the intraluminal medical device are implantable, assemblable, and operable intraluminally. The intraluminal dock includes a pump dock, and the intraluminal medical device includes a pump. Also disclosed are methods of implantation and explantation of the intraluminal modular powered medical system.

### CROSS-REFERENCE TO RELATED APPLICATIONS

The present patent application is a continuation of International Patent Application No. PCT/CA2023/050378, filed Mar. 22, 2023, which claims the benefit of and priority to U.S. Provisional Patent Application No. 63/322,691, filed Mar. 23, 2022, the entire contents of which, including all references incorporated by reference therein, if any, are incorporated herein by reference for all purpose as if fully set forth herein, except for any definition(s), subject matter disclaimer(s) or disavowal(s), and except to the extent that the incorporated material is inconsistent with the express disclosure herein, in which case the language in the present disclosure controls.

### FIELD

The present disclosure generally relates to an intraluminal modular powered medical system.

### BACKGROUND

Ventricular assist devices (VADs) generally come as left ventricular assist devices (LVAD), right ventricular assist devices (RVAD), or biventricular assist devices (BiVAD). Multiple designs of VADs are known, including intraluminal modular powered medical systems provided with multiples pumps.

In order to provide hemodynamic support, VADs are called on to displace large amounts of blood, for example in the range of one to five liters of blood per minute, and as such, often require significant amounts of energy to operate properly. Accordingly, implanted VADs are typically wired to a power source that is located outside a subject's vasculature, requiring a powerline to extend between the VAD and the power source.

The powerline often encloses one or more electrical cable(s) or electrical wire(s) to transmit electric power to one or more of the pump(s) and/or one or more mechanical driveline to transmit torque to one or more pump(s). As such, the cross-sectional size of the powerline may be a substantial fraction of the lumen of the subject's vasculature in which the VAD is implanted, and thus the powerline may hinder blood flow in that lumen. This is especially true when the lumen of the subject's vasculature progressively reduces downstream of blood flow. The bigger the cross-sectional area of the powerlines(s), the more the blood flow is hindered. Similarly, the more powerlines in a lumen of a vasculature, the more the blood flow is hindered. For example, multi-pump VAD designs may require each pump to be wired to a power source, resulting in multiple powerlines, either individually or as a bundle, passing through the vasculature to transmit power from the power source to the pumps.

Decrease or even complete loss of blood flow downstream of the powerline may lead to tissue ischemia and/or other complications. Ischemia, even if only temporary, may cause irreversible damage to tissues. For example, a few hours of blood flow occlusion in the femoral artery by a VAD implanted via a transfemoral approach may lead to lower limb ischemia and ultimately may result in lower limb amputation. Along with an aging population comes an increased atherosclerotic burden in peripheral vessels, resulting in smaller diameter vessel lumens, thereby further complicating matters.

Therefore, improvement(s) over at least some known VADs are desirable.

### SUMMARY

A first embodiment on the present disclosure is directed to an intraluminal modular powered medical system, including:
an intraluminal dock including at least one dock electrical connector configured to receive power from a power source; and
an intraluminal medical device including at least one device electrical connector configured to intraluminally electrically connect to the at least one dock electrical connector for receiving power therefrom.

The intraluminal medical device may include a control element configured to attached thereto and to extend therefrom, the control element may be further configured to be actuated for connecting the at least one device electrical connector and the at least one dock electrical connector together.

The control element may be configured to removably attach to the intraluminal medical device.

The control element may be configured to extend from the at least one device electrical connector.

The control element may be further configured to be in a slidable relationship with the intraluminal dock, the control element may be further configured to be slidably actuated for connecting the at least one device electrical connector and the at least one dock electrical connector together.

The intraluminal dock may include a control element passage guide, the control element passage guide may be configured to slidably receive the control element therealong for connecting the at least one device electrical connector and the at least one dock electrical connector together.

The control element may have a proximal end portion that is configured to be manipulated extracorporeally by an operator for connecting the at least one dock electrical connector and the at least one device electrical connector together.

The control element may be configured to be pulled for connecting the at least one device electrical connector to the at least one dock electrical connector.

The control element may be configured to be pushed for connecting the at least one device electrical connector to the at least one dock electrical connector.

The at least one device electrical connector and the at least one dock electrical connector may be configured to removably connect together, the control element may be further configured to be pulled for disconnecting the at least one device electrical connector from the at least one dock electrical connector when connected thereto.

The at least one device electrical connector and the at least one dock electrical connector may be configured to removably connect together, the control element may be further configured to be pushed for disconnecting the at least one device electrical connector from the at least one dock electrical connector when connected thereto.

The intraluminal dock may include a dock physical connector, and the intraluminal medical device may include a device physical connector configured to intraluminally connect to the dock physical connector.

The control element may be a guide wire.

The control element passage guide may be a guide hole.

The intraluminal dock may include an intraluminal extension configured to attached thereto and to extend therefrom.

The intraluminal extension may include an electrical conductor configured to connect to a power source for supplying power to the at least one dock electrical connector.

The intraluminal dock may include a receiver coil operatively connected to the at least one dock electrical connector and configured to wirelessly receive power from a power source.

The intraluminal extension may include a longitudinal channel configured to slidably receive the control element therein.

The intraluminal extension may include a longitudinal channel configured to circulate fluid therein for fluid delivery to the pump dock and outside the pump dock.

The intraluminal extension may include a longitudinal channel configured to receive a stiff guide wire therein for increasing stiffness of the intraluminal extension.

The intraluminal extension may include a longitudinal channel configured to be in communication with the control element passage guide.

The intraluminal extension may be configured to be actuated for connecting the at least one dock electrical connector and the at least one device electrical connector together.

The intraluminal extension may have a proximal end portion that is configured to be manipulated extracorporeally by an operator for connecting the at least one dock electrical connector and the at least one device electrical connector together.

The intraluminal extension may be configured to be pulled for connecting the at least one dock electrical connector to the at least one device electrical connector.

The intraluminal extension may be configured to be pushed for connecting the at least one dock electrical connector to the at least one device electrical connector.

The at least one dock electrical connector and the at least one device electrical connector may be configured to removably connect together, the intraluminal extension may be further configured to be pulled for disconnecting the at least one dock electrical connector from the at least one device electrical connector when connected thereto.

The at least one dock electrical connector and the at least one device electrical connector may be configured to removably connect together, the intraluminal extension may be further configured to be pushed for disconnecting the at least one dock electrical connector from the at least one device electrical connector when connected thereto.

The intraluminal dock may be configured to intraluminally dock at least partially to the intraluminal medical device when the at least one dock electrical connector is connected to the at least one device electrical connector.

The intraluminal dock may include a pump receiving surface configured to mate at least partially with the intraluminal medical device, on an outer face thereof, when the at least one dock electrical connector is connected to the at least one device electrical connector.

The at least one dock electrical connector may define a cavity, and the system may further include a plug configured to fluidically sealingly engage the cavity.

The control element may be routed through the plug.

The intraluminal dock may include an anchor configured to intraluminally anchor the intraluminal modular powered medical system.

The system may include: an intraluminal dock including a plurality of dock electrical connectors, each dock electrical connectors may be configured to receive power from a power source; and a plurality of intraluminal medical devices, each intraluminal medical devices may include a respective device electrical connector that may be configured to intraluminally electrically connect to a corresponding connector of the plurality of dock electrical connectors for receiving power therefrom; each connector of the plurality of device electrical connectors and each connector of the plurality of dock electrical connectors may be configured to correspondingly connect together (i) simultaneously and (ii) in a stepwise manner.

The intraluminal medical device may be a pump, and the intraluminal dock may be a pump dock.

A second embodiment on the present disclosure is directed to an intraluminal modular powered medical system, including:
an intraluminal powerable medical device including at least one control wire; and
an intraluminal control element guide including at least one guide hole that is sized and shaped to receive the control guide therealong, the intraluminal control element guide being configured to assemble with the intraluminal powerable medical device by control wire actuation and also being configured to power the intraluminal powerable medical device when assembled therewith.

A third embodiment on the present disclosure is directed to a method of implanting an intraluminal modular powered medical system to an intraluminal implantation site in a lumen of a subject, the method including:
delivering the intraluminal modular powered medical system to the intraluminal implantation site, the intraluminal modular powered medical system including an intraluminal dock having a dock electrical connector, and an intraluminal medical device having a device electrical connector that is electrically connectable to the dock electrical connector; and
intraluminally connecting the dock electrical connector and the device electrical connector together for powering the intraluminal medical device.

Intraluminally connecting may include: manipulating a control element of the intraluminal medical device to intraluminally connect the device electrical connector and the dock electrical connector together for powering the intraluminal medical device.

Manipulating the control element may include: slidably manipulating the control element.

Manipulating the control element may further include: manipulating a proximal end portion of the control element that is located extracorporeally.

Intraluminally connecting may further include: manipulating an intraluminal extension of the intraluminal dock to intraluminally connect the dock electrical connector and the device electrical connector together for powering the intraluminal medical device.

Manipulating the intraluminal extension may include: slidably manipulating the intraluminal extension.

Manipulating the intraluminal extension may further include: manipulating a proximal end portion of the intraluminal extension that is located extracorporeally.

The method may further include: supplying power to the intraluminal medical device via the intraluminal dock by at least one of (i) electrically connecting the intraluminal dock to a power source; and (ii) wirelessly transferring energy between the intraluminal dock and a power source.

Delivering the intraluminal modular powered medical system may include: obtaining an intraluminal access opening for delivering the intraluminal modular powered medical system therethrough to the intraluminal implantation site.

Delivering the intraluminal modular powered medical system may further include: introducing a sheath containing at least partially the intraluminal modular powered medical system therein in the lumen through the intraluminal access opening; intraluminally navigating the sheath through the lumen up to the intraluminal implantation site thereof, and exiting the modular intraluminal medical device assembly from the sheath in the lumen at the intraluminal implantation site thereof.

The method may further include: anchoring the intraluminal modular powered medical system at the intraluminal implantation site.

Delivering the intraluminal modular powered medical system may further includes: removing a sheath from the lumen after the intraluminal modular powered medical system has been exited therefrom at the intraluminal implantation site.

Delivering the intraluminal modular powered medical system may further include: chirurgicaly closing the intraluminal access opening.

The intraluminal medical device may be an intraluminal medical device, and the intraluminal dock may be an intraluminal dock.

A fourth embodiment on the present disclosure is directed to a method of explanting an intraluminal modular powered medical system from an intraluminal implantation site in a lumen of a subject, the intraluminal modular powered medical system including an intraluminal dock having a dock electrical connector and an intraluminal medical device having a device electrical connector that is electrically disconnectable from the dock electrical connector, the method including:
intraluminally disconnecting the dock electrical connector and the device electrical connector from one another for unpowering the intraluminal medical device; and
retrieving the intraluminal modular powered medical system from the intraluminal implantation site.

Intraluminally disconnecting may include: manipulating a control element of the intraluminal medical device to intraluminally disconnect the device electrical connector and the dock electrical connector from one another for unpowering the intraluminal medical device.

Manipulating the control element may include: slidably manipulating the control element.

Manipulating the control element may further include: manipulating a proximal end portion of the control element that is located extracorporeally.

Intraluminally disconnecting may further include: manipulating an intraluminal extension of the intraluminal dock to intraluminally disconnect the dock electrical connector and the device electrical connector from one another for unpowering the intraluminal medical device.

Manipulating the intraluminal extension may include: slidably manipulating the intraluminal extension.

Manipulating the intraluminal extension may further include: manipulating a proximal end portion of the intraluminal extension that is located extracorporeally.

The method may further include: interrupting the supply of power to the intraluminal medical device by at least one of (i) electrically disconnecting the intraluminal dock from a power source; and (ii) interrupting a wirelessly transferring energy between the intraluminal dock and the power source.

Retrieving the intraluminal modular powered medical system may include: obtaining an intraluminal access opening for retrieving the intraluminal modular powered medical system therethrough from the intraluminal implantation site.

Retrieving the intraluminal modular powered medical system may further include: introducing a sheath in the lumen through the intraluminal access opening; intraluminally navigating the sheath through the lumen up to the intraluminal implantation site thereof, and taking up at least partially the modular intraluminal medical device system in the sheath from the lumen at the intraluminal implantation site thereof.

The method may further include: unanchoring the intraluminal modular powered medical system from the intraluminal implantation site.

Retrieving the intraluminal modular powered medical system may further include: removing a sheath containing at least partially the intraluminal modular powered medical system therein from the lumen.

Retrieving the intraluminal modular powered medical system may further include: chirurgicaly closing the intraluminal access opening.

The intraluminal medical device may be a pump, and the intraluminal dock may be an intraluminal dock.

### Definitions

As intended herein:

The terms “proximal” and “proximally” refer to a location or a position that is closer to an operator of the intraluminal modular powered medical system described herein, as compared to a location or a position that is distal to, or distally positioned or distally located to, this same operator.

The terms “distal” and “distally” refer to a location or a position that is farther to an operator of the intraluminal modular powered medical system described herein, as compared to a location or a position that is proximal to, or proximally positioned or proximally located to, this same operator.

The terms “comprising,” “having,” “including,” and “containing” are to be construed as open-ended terms.

### BRIEF DESCRIPTION OF THE DRAWINGS

FIG. 1A is a schematic diagram of a pump dock of a modular pump system, according to an embodiment.

FIG. 1B is a schematic diagram of a first pump and a second pump of a modular pump system, where both the first pump and the second pump are dockable to the pump dock of FIG. 1A.

FIG. 1C is a schematic diagram of a modular pump system, where both the first pump and the second pump of FIG. 1B are docked to the pump dock of FIG. 1A.

FIG. 2A is a flow chart showing a sequence of operations for implanting a modular pump system, according to an embodiment.

FIG. 2B is a flow chart showing a sequence of operations for explanting a modular pump system, according to an embodiment.

FIGS. 3A-3D, 4A-4D, and 5A-5C show a modular pump system provided with a plurality of pumps, where each pump has a respective prong electrical connector, according to an embodiment.

FIGS. 6A-E, 7A-7D, and 8A-8C show a modular pump system that includes a plurality of pumps, where each pump has respective prong electrical connector and a respective fluid seal, according to an embodiment.

FIGS. 9A-9E, 10A-10D, and 11A-11C show a modular pump system that includes a plurality of pumps, where each pump has a respective prong electrical connector and two respective fluid seals, according to an embodiment.

FIGS. 12A-12D, 13A-13D, and 14A-14C show a modular pump system that includes a plurality of pumps, where each pump has multiple respective prong connectors, according to an embodiment.

FIGS. 15A-15D, 16A-16D, and 17A-17C show a modular pump system that includes a plurality of pumps, where each pump has a respective prong connector having protruded conductive sections circumferentially distributed thereabout, according to an embodiment.

FIGS. 18A-18D, 19A-19D, and 20A-20C show a modular pump system that includes a plurality of pumps and a plurality of control elements, where each control element is removably attachable to a corresponding pump, according to an embodiment.

FIGS. 21A-21D, 22A-22C, and 23A-23D show a modular pump system that includes a plurality of pumps, a plurality of control elements, and a plurality of catheters, where each catheter is attached to a corresponding pump and defines a lumen to house a corresponding control element therein that is removably attachable to a corresponding pump, according to an embodiment.

FIGS. 24A-24C and 25A-25D show a modular pump system configured to fluidically couple to a fluid flush device, according to an embodiment.

FIGS. 26A-26D show a modular pump system having a coil configured to wirelessly receive power, according to an embodiment.

FIGS. 27A-27D and FIGS. 28A-28C show a modular pump system having a coil configured to wirelessly receive power, according to an embodiment.

FIGS. 29A-29D show a modular pump system that includes a plurality of pumps, where each pump is configured to be pushed in order to connect and dock the pumps to a pump dock, according to an embodiment.

### DETAILED DESCRIPTION

The modular pump systems of the present disclosure are assemblable and operable in vivo. As described hereinafter, each of the modular pump systems can be transcatheterally and/or percutaneously delivered in an unassembled, undocked configuration to an intraluminal site for implantation in a subject's vasculature. Then, each of the modular pump systems can be converted from the unassembled, undocked configuration to an assembled, docked configuration and powered for operation of the pumps. Each of the modular pump systems can also be transcatheterally and/or percutaneously retrieved from an intraluminal site for explantation from a subject's vasculature after having been converted from the assembled, docked configuration to the unassembled, undocked configuration, and the pumps having been unpowered. In the unassembled, undocked configuration the pumps are generally arranged in serial or one after the other, while in the assembled, docked configuration the pumps are generally arranged in parallel or one next to each other. Intraluminal sites for implantation include any lumen of the body, such as the vasculature, including the chambers of the heart, hollow organs, and body cavities.

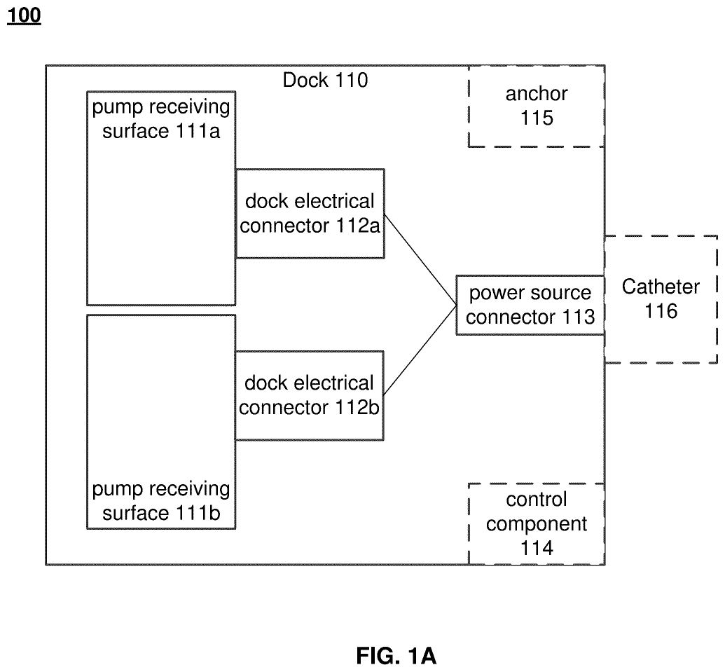

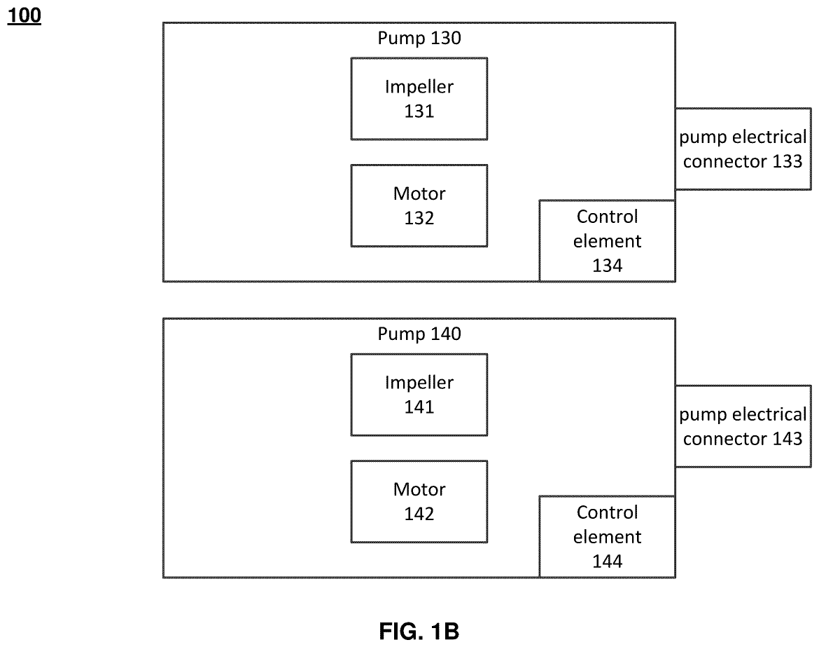

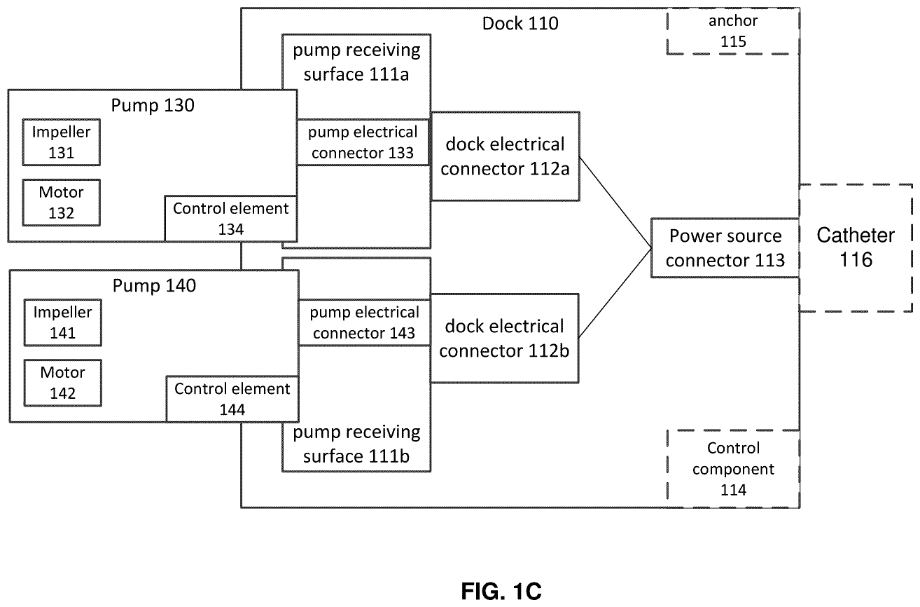

FIGS. 1A to 1C are schematic diagrams of a modular pump system 100 (also referred to herein as an intraluminal modular powered medical system) and components thereof. In FIGS. 1A to 1C, solid lines indicate elements of the modular pump system 100, and dotted lines indicate optional elements of the modular pump system 100. More specifically, FIG. 1A schematically shows a pump dock 110 (also referred to herein as an intraluminal dock or an intraluminal control element guide), according to an embodiment. FIG. 1B schematically shows first and second pumps 130, 140 (also referred to herein as first and second intraluminal medical devices or first and second intraluminal powerable medical devices), according to an embodiment. FIG. 1C schematically shows the first and second pumps 130, 140 docked to the pump dock 110 (that is, in a docked configuration), according to an embodiment. While FIGS. 1A to 1C are directed to the modular pump system 100 and components thereof, it will be appreciated that FIGS. 1A to 1C also apply, with the necessary change(s) appreciable to the skilled addressee having been made, if applicable, to the modular pump systems 300, 400, 500, 600, 700, 800, 900, 1000, 1100, 1200, 1300 and their respective components thereof, as described herein.

The pump dock 110 provides a structure to which at least one pump is dockable and electrically connectable thereto to be powered and operated. The pump(s) may be removably dockable (i.e., undockable) and removably electrically connectable (i.e., electrically disconnectable) to the pump dock 110. FIG. 1A shows the pump dock 110 including first and second dock electrical connectors 112a, 112b, whereas FIG. 1C shows the pump dock 110 including first and second dock electrical connectors 112a, 112b electrically connected to first and second pumps 130, 140, respectively. More specifically, the first dock electrical connector 112a is configured to be mechanically and electrically connected to the first pump 130 via a first pump electrical connector 133 (also referred to herein as a device electrical connector; described hereinafter), thereby docking the first pump 130 to the pump dock 110. Similarly, the second dock electrical connector 112b is configured to be mechanically and electrically connected to the first pump 140 via a second electrical connector 143 (also referred to herein as a device electrical connector; described hereinafter), thereby docking the second pump 140 to the pump dock 110. The first and second dock electrical connectors 112a, 112b may be electrically disconnectable from the first and second pump electrical connectors 133, 143, respectively, so that the first and second pumps 130, 140 may be undockable from the pump dock 110, respectively.

Hence, the first and second dock electrical connectors 112a, 112b may not be configured only to provide electricity to the corresponding first and second pump electrical connectors 133, 143 when connected thereto, but also to dock the pump dock 110 to the first and second pumps 130, 140.

The pump dock 110 may include more than one dock electrical connector per pump connectable thereto.

The pump dock 110 may include at least one dock physical connector (not shown in FIGS. 1A and 1C) per pump connectable thereto. The dock physical connector, which does not provide electricity to the pump, may be: (i) configured to engage and/or maintain, in combination with the dock electrical connector, a physical docking interaction between the pump dock 110 and a pump (i.e., the physical connection component of the docking interaction between the pump dock 110 and the pump is shared by the dock physical connector and the dock electrical connector); or (ii) configured to engage and/or maintain, without the assistance of the dock electrical connector, a physical docking interaction between the pump dock 110 and a pump (i.e., the physical connection component and the electrical connection component of the docking interaction between the pump dock 110 and the pump is separated between the dock physical connector and the dock electrical connector, respectively). For example, the pump dock 110 may include one dock electrical connector and one dock physical connector.

The pump dock 110 includes at least one control element passage guide (also referred to herein as a guide hole) that is defined therealong or therethrough and that is sized and shaped to receive at least one corresponding control element therein, such as a first control element 134 of a first pump 130 or a second control element 144 of a second pump 140 (shown in FIG. 1B). So received, the control element may be actuated to slidably move along the control element passage guide, resulting in a movement of the corresponding pump relative to the pump dock 110. For example, the pump dock 110 may include first and second control element passage guides (not shown), each sized and shaped to receive corresponding first and second control elements 134, 144 of first and second pumps 130, 140 therealong, respectively. Actuation of the first and second control elements 134, 144 along the first and second control element passage guides, respectively, may slidably move the first and second pump 130, 140 relative to the pump dock 110, respectively.

The pump dock 110 may include multiple control element passage guides, each being sized and shaped to receive one corresponding control element of one of multiple pumps. Alternatively, the pump dock 110 may include only one control element passage guide that is sized and shaped to receive all the corresponding control elements of multiple respective pumps.

The pump dock 110 may optionally include at least one pump receiving surface that is sized and shaped to mate with a corresponding pump, such as on an outer face thereof. The pump receiving surface and the pump may be complementary in size and/or shape in order to structurally conform to one another when mated together. The pump receiving surface may at least help in guiding, engaging, and/or maintaining a docking interaction between the pump dock 110 and a pump to be docked or already docked to the pump dock 110. Mating between the pump receiving surface and the pump may be a removable mating.

As the pump and the pump dock 110 are getting closer to one another, such as by sliding the pump toward the pump dock 110, the pump progressively engages and/or contacts a corresponding pump receiving surface. As the pump progress along the pump receiving surface, the pump electrical connector progressively engages the dock electrical connector and eventually establish an electrical connection therewith.

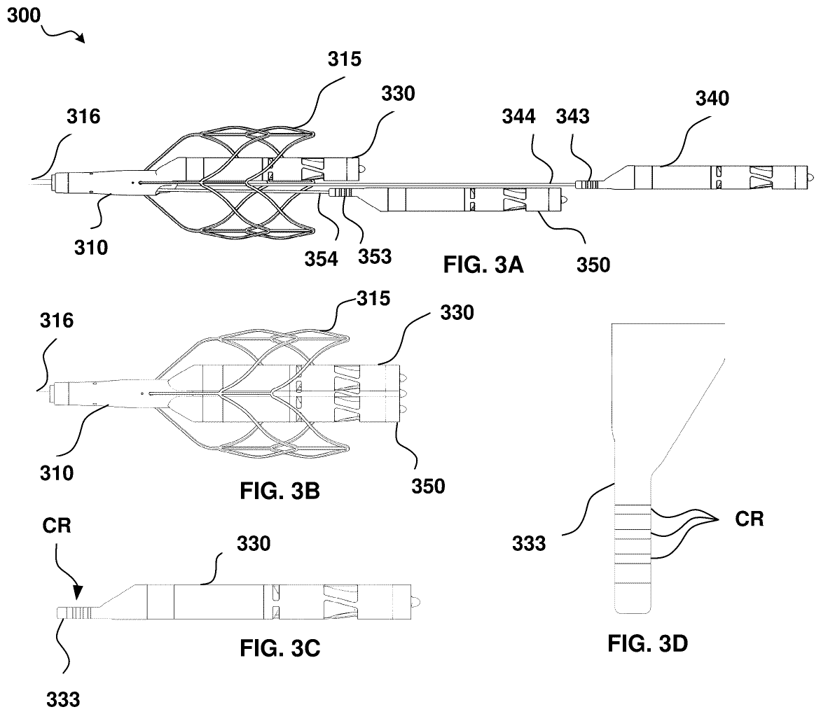

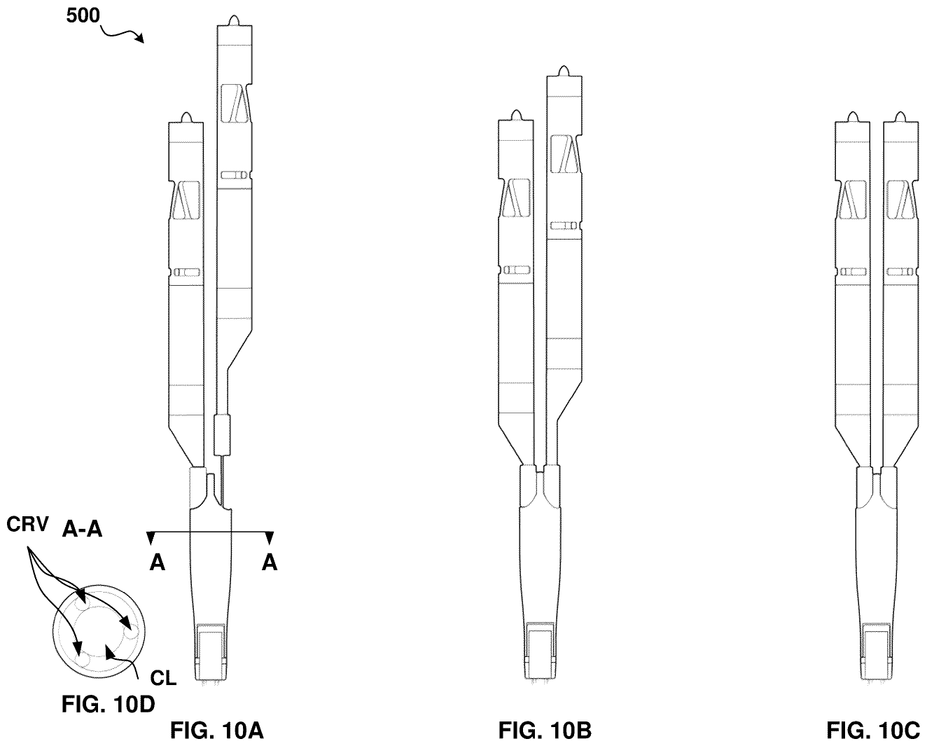

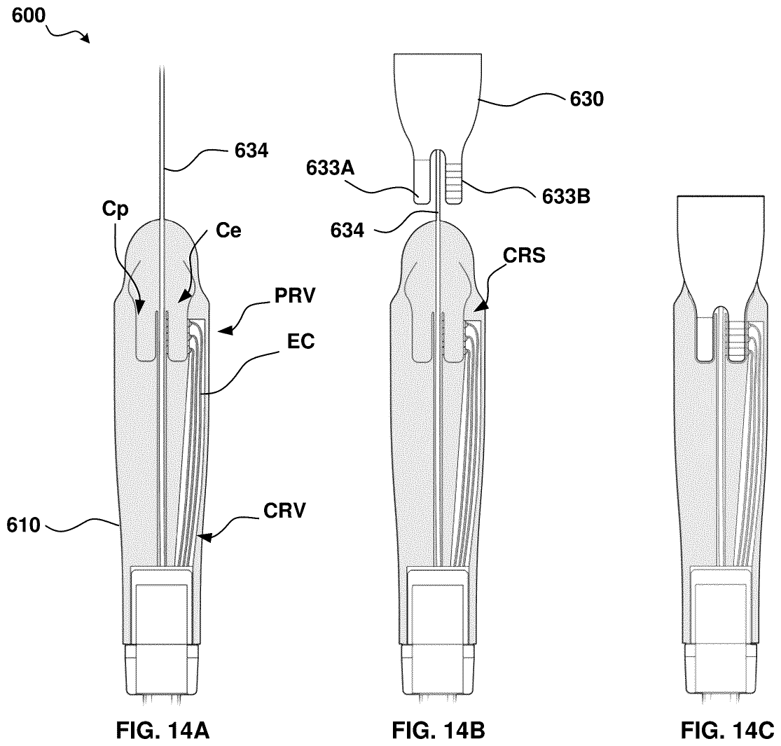

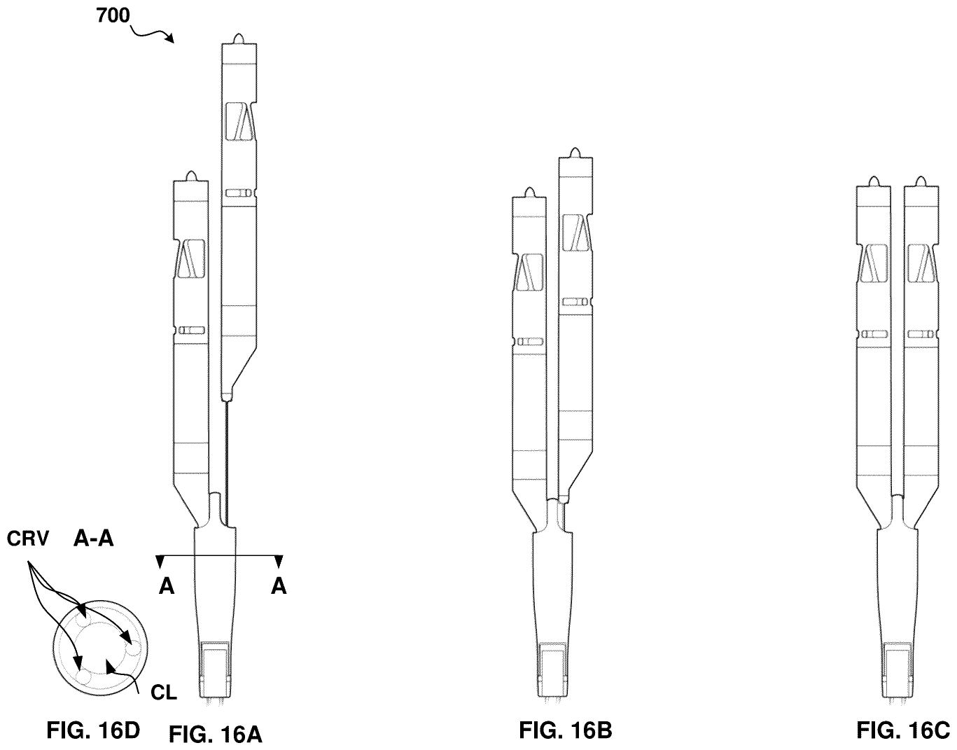

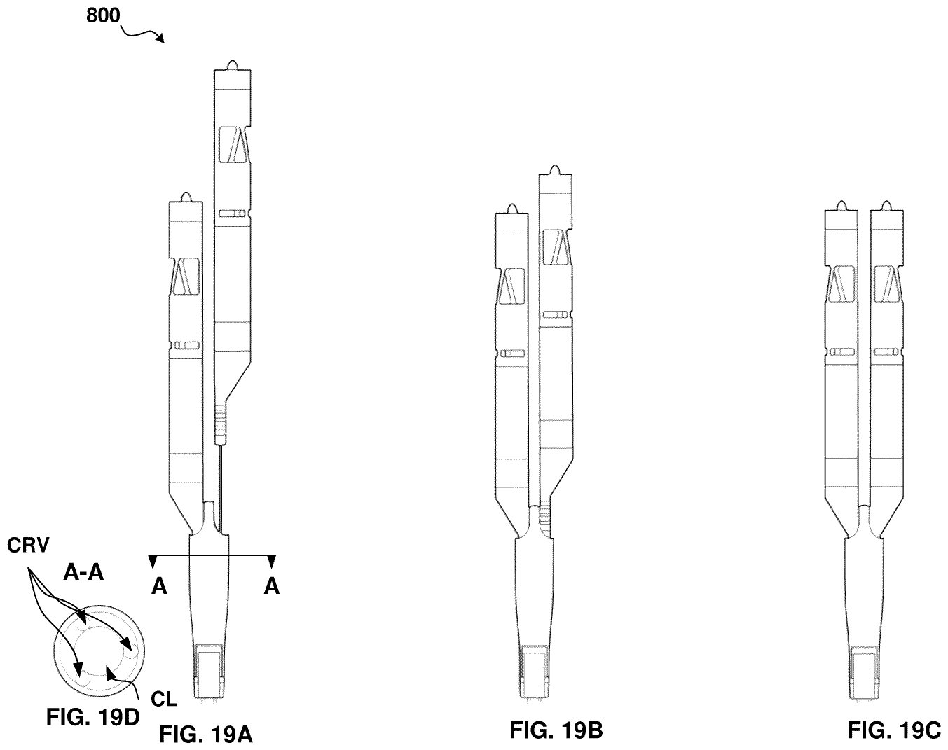

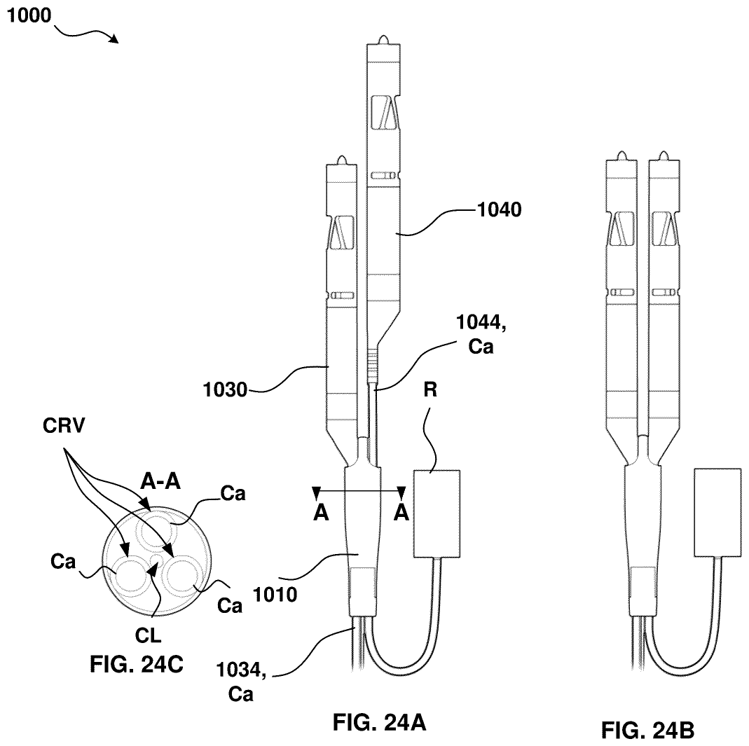

As shown in FIGS. 1A and 1C, the pump dock 110 may include a first pump receiving surface 111a that is associated with or integrated to the first dock electrical connector 112a, and a second pump receiving surface 111b associated with or integrated to the second dock electrical connector 112b. As further shown in the embodiments of FIGS. 3A, 4A, 5A, 6A, 7A, 8A, 9A, 10A, 11A, 12A, 13A, 14A, 15A, 16A, 17A, 18A, 19A, 20A, 21A, 22A, 23c, 24A, 25A, for example, the pump receiving surfaces 111a, 111b are formed at least partially by a projection projecting from the pump dock in a distal direction (i.e., in a direction opposite to the power source connector, described hereinafter, extending from the pump dock). As such, the first and second dock electrical connectors 112a, 112b are associated with or integrated to the pump receiving surfaces 111a, 111b, respectively. The first and second dock electrical connectors 112a, 112b generally define a respective cavity extending in a proximal direction, opposite to the distal direction of the projection.

When multiple pumps are connectable to the pump dock 110, the projection may be positioned and arranged on the pump dock 110 to define one or more pump receiving surface(s) that is/are sized and shaped to mate with only a subset of multiple corresponding pumps, while the remaining pump(s) connect to the pump dock 110 without mating with any pump receiving surface.

One or more pump receiving surface(s) may be associated to or integrated with one or more dock electrical connector(s) and/or one or more dock physical connector(s). When not associated to or integrated with a pump receiving surface, the electrical connector(s) and/or dock physical connector(s) may be located elsewhere on the pump dock 110.

As shown in FIGS. 1A and 1C, the pump dock 110 may further include at least one power source connector 113 (also referred to herein as an intraluminal extension) that projects from the pump dock 110 such that, when the modular pump system 100 is delivered or implanted, the power source connector 113 may extend intraluminally, such as through the vasculature, and be connected to a power source (not shown).

The power source connector 113 is connected, at a distal end portion thereof, to the first and second dock electrical connectors 112a, 112b and is configured to deliver power thereto, and consequently to the first and second pumps 130, 140, respectively, when connected to the pump dock 110. As such, the power source connector may include an electrical conductor, an electrical cable, or an electrical wire. The proximal end portion of the power source connector 113 may be disposed intraluminally (e.g., in the vasculature), extraluminally (e.g., outside the vasculature but still in a subject's body), and extracorporeally. The proximal end portion of the power source connector 113 is manipulable, either intraluminally or extracorporeally, by an operator in order to intraluminally move and dock and connect the pump dock 110 to the first and second pumps 130, 140 and undock and disconnect the pump dock 110 from the first and second pumps 130, 140.

The proximal end portion of the power source connector 113 is operatively connectable or removably operatively connectable to a power source via an electrical wired connection or a wireless power transfer. In the case of an electrical wired connection, the power source connector 113 is operatively connectable to the power source intraluminally (e.g., in the vasculature), extraluminally (e.g., outside the vasculature but still in a subject's body), and extracorporeally. In the case of a power wireless transfer, the power source connector 113 is electrically connected to a receiver coil (not shown) that is configured to receive power from a transmitter coil (not shown) of a power source.

Alternatively, the power source connector 113 may be itself a receiver coil in the shape of a coil, spiral, helix, etc. that is coupled to the pump dock 110 at any suitable location, such as to the anchor 115, and configured to receive power from a transmitter coil of a power source. The wireless power transfer may include a near-field inductive power transfer (e.g., when in use the transmitter coil and the receiver coil are located intracorporeally or extracorporeally) or a transcutaneous near-field inductive energy transfer (e.g., when in use the transmitter coil is located extracorporeally, and the receiver coil is located intracorporeally.

Still alternatively or additionally, the pump dock 110, which may be configured to receive power from the power source via a wire or wirelessly, includes a transmitter coil, and the pump(s) includes a receiver coil configured for wirelessly receiving power from the transmitter coil of the pump dock 110.

The pump dock 110 may also include a battery, such as a rechargeable battery, configured to receive power from a power source and to supply power to at least one of the pumps 130, 140.

Advantageously, the wireless transmission of power from outside of the body to the modular pump system 100 enables an improved design where there is no need to extend a power cable or wire from the pump dock 110 through and/or out of the subject's body to power and/or control the modular pump system 100. This in turn at least reduces the risk associated with infection, hemostasis and other related complications.

The power source includes any type of suitable electrical power sources known in the art, including one or more battery, a power grid or any other suitable energy supply and/or energy storage, and combination thereof. The power source may be configured to deliver power to the pump dock via a wire and/or wirelessly.

As shown in FIGS. 1A and 1C, the pump dock 110 may further include at least one control component 114 (also referred to herein as an intraluminal extension) that extends from the pump dock 110 such that, when the modular pump system 100 is delivered or implanted, the control component 114 may extend intraluminally, such as through the vasculature. As such, the proximal end portion of the control component 114 may be disposed intraluminally (e.g., in the vasculature), extraluminally (e.g., outside the vasculature but still in a subject's body), and extracorporeally.

The control component 114 may be a guide wire or any other suitable wire, cable, and the like (in this case, also referred to herein as a wire control component) that is manipulable, either intraluminally or extracorporeally, at a proximal end portion thereof by an operator to dock and connect the pump dock 110 to the first and second pumps 130, 140 and to undock and disconnect the pump dock 110 from the first and second pumps 130, 140. As such, the control component 114 may be attachable to and detachable from the pump dock 110 by manipulation by an operator. For example, the wire control component may be screwable to the pump dock 110 so that the operator may rotate the wire control component to screw the wire control component to the pump dock 110 and unscrew the wire control component from the pump dock 110. The control component 114 may also be a snare configured to capture and/or release the pump dock 110. When attached, the wire control component 114 may be used to pull and/or push the modular pump system, including the pump dock 110.

The control component 114 may also be a catheter (in this case, also referred to herein as a catheter control component) configured to carry component(s) and/or fluid(s) to the pump dock 110 and thus to optionally deliver same intraluminally. For example, a relatively stiff guide wire (also referred to herein as a stiff guide wire) may be routed in the catheter control component to provide structural rigidity and or tactile feedback to the catheter control component for improving intraluminal navigation of the pump dock 110 (docked to or undocked from the pump(s)), such as in the vasculature. For example, such modulable and improved structural rigidity and/or stiffness of the catheter control component may be useful when the pump dock 110 needs to be pushed toward the first and second pumps 130, 140 by an operator, such as for docking and/or undocking purpose(s). Indeed, the improved structural rigidity and/or stiffness provided by the stiff guide wire to the catheter control component may better transmit the pushing force to the pump dock 110 instead of being inappropriately bent or folded by such force to impede or prevent the manipulation intended by the operator.

Selection of wires of various stiffness and/or insertion and removal of such wires(s) at least partially in and out the catheter control component may modulate the structural rigidity and/or stiffness of the catheter control component and thus further improve intraluminal navigation and tactile feedback of the pump dock 110.

The catheter control component may also be used with a fluid flush device (not shown) to provide flush solution (e.g., dextrose flush) to the pump dock 110. For example, the fluid flush device may provide a fluid at a high pressure (e.g., at a higher pressure than the intraluminal or intracavity pressure in which the device is implanted, such as >140 mm Hg in the arterial circulation) from the pump dock 110 to the pump(s) via a fluid connector, which may be structurally integrated to the pump electrical connector(s) 133 and/or 143, to prevent blood from entering the motor of the pumps 130 and/or 140. The fluid flush device may also provide fluid to the pump(s) 130 and/or 140 in a closed circuit to hydraulically entrain rotation of the impeller of the pump (e.g., in instance where the motor is not an electric motor).

As shown in FIGS. 1A and 1C, the pump dock 110 may further include at least one catheter 116 (also referred to herein as an intraluminal extension) that projects from the pump dock 110 such that, when the modular pump system 100 is delivered or implanted, the catheter 116 may extend intraluminally, such as through the vasculature. As such, the proximal end portion of the catheter 116 may be disposed intraluminally (e.g., in the vasculature), extraluminally (e.g., outside the vasculature but still in a subject's body), and extracorporeally. The proximal end portion of the catheter 116 is manipulable, either intraluminally or extracorporeally, by an operator in order to intraluminally move and dock and connect the pump dock 110 to the first and second pumps 130, 140 and undock and disconnect the pump dock 110 from the first and second pumps 130, 140.

The catheter 116 defines at least one longitudinal channel (also referred to herein as a lumen; not shown) therethrough along at least a portion thereof. The catheter 116 is sized and shaped to receive the power source connector 113, the control component 114, and/or the first and second control elements 134, 144 therein (also collectively referred to herein as intraluminal extensions). All the intraluminal extensions may be received in a single longitudinal channel. Alternatively, the intraluminal extensions, or combination thereof, may be received in a respective longitudinal channel. For example, the catheter 116 may define two longitudinal channels, each configured for receiving one of the first and second control elements 134, 144 therein. The longitudinal channels may be sized and/or shaped in any suitable manner. For example, the cross-sectional size of a longitudinal channel may be sized to be just slightly larger than the cross-sectional size of a given control element to allow for sufficient translation of the control element therethrough, while limiting any free space in which control element could buckle, twist, etc.

As shown in FIGS. 1A and 1C, the pump dock 110 may further include an anchor 115 configured to anchor the pump dock 110 (and the modular pump system 100 when the first and second pumps 130, 140 are docked to the pump dock 110) intraluminally, such as at an intraluminal implantation site in the aorta or in a chamber of the heart. For example, the anchor may secure the pump dock 110 to a wall of the vasculature by applying a force thereto. The pump dock 110 has a compact configuration for intraluminal delivery, such as by a transcatheter procedure, and an expanded configuration for anchoring the pump dock 110 intraluminally. The cross-sectional size of the anchor in the compact configuration is smaller than the cross-sectional size of the anchor 115 in the expanded configuration.

The anchor 115 may be self-expandable (e.g., made of a memory material like Nitinol), such that the anchor converts from the compact configuration to the expanded configuration at the intraluminal implantation site by itself (e.g., by being exited from a sheath or sheath). When self-expandable, the anchor 115 may be biased or overcomeably biased toward the expanded configuration. For example, the anchor may be as the anchor assembly described in International Patent Application Number PCT/ZA2020/050,022, which is incorporated herein by reference in its entirety, including all reference incorporated by reference therein, for all purpose as if fully set forth herein, except for any definition, subject matter disclaimer or disavowals, and except to the extent that the incorporated material is inconsistent with the express disclosure herein, in which case the language in the present disclosure controls.

Alternatively, the anchor 115 may also be expandable mechanically. For example, a balloon catheter disposed within the anchor 115 may be inflated to partially or completely convert the anchor 115 from the compact configuration to the expanded configuration (in this case the anchor 115 is made of a “non-memory” material).

Still alternatively, the anchor 115 may be operably coupled to an anchor actuation stem (not shown) configured to be manipulated by an operator to convert or cause the conversion of the anchor 115 from the compact configuration to the expanded configuration (in this case, the anchor 115 may be made of a memory material biasing the anchor 115 toward the expanded configuration) and/or from the expanded configuration to the compact configuration.

The anchor 115 may be releasably coupled to the pump dock 110 and thus be releasable from the pump dock 110 upon actuation of an actuation rod or actuation wire, as described for the mammalian body conduit intralumenal device and lumen wall anchor assembly described in International Patent Application Number PCT/US2021/012,083, which is incorporated herein by reference in its entirety, including all reference incorporated by reference therein, for all purpose as if fully set forth herein, except for any definition, subject matter disclaimer or disavowals, and except to the extent that the incorporated material is inconsistent with the express disclosure herein, in which case the language in the present disclosure controls.

As show in FIG. 1B, the modular pump system 100 includes at least one pump (also referred to herein as an intraluminal medical device and an intraluminal powerable medical device), such as first and second pumps 130, 140. The first pump 130 (and components thereof, if applicable) being similar to the second pump 140 (and components thereof, if applicable), only the first pump 130 (and components thereof, if applicable) will be described hereinafter. The skilled addressee will appreciate that the description of the first pump 130 (and components thereof, if applicable) applies to the second pump 140 (and components thereof, if applicable) with the necessary change(s), appreciable to the skilled addressee, having been made, if applicable.

The first pump 130 includes a first impeller 131 for moving fluid intraluminally; a first motor 132 operatively coupled, either directly or indirectly (e.g., via a gearbox), to the first impeller 131 for rotating same; a first pump electrical connector 133 (also referred to herein as first device electrical connector) operatively connected to the first motor 132 for delivering power thereto and operatively connectable or removably operatively connectable to the first dock electrical connector 112a for receiving power therefrom; and a first control element 134 (also referred to herein as a intraluminal extension) attached to the first pump 130 and projecting away therefrom for intraluminally moving the pump 130. The first pump 130 is configured to be intraluminally delivered undocked from the pump dock 110 (i.e., in the unassembled, undocked configuration) and to be intraluminally docked to the pump dock 110 (i.e., in the assembled, docked configuration), such as in a subject's vasculature.

The first impeller 131 may be an axial impeller, a peripheral impeller, a mixed flow impeller, or a radial impeller. Preferably, the first impeller is an axial impeller of an axial-flow pump.

The first motor 132 may be an electric motor, such as brushless electric motor.

As shown in FIG. 1B, the first pump 130 may include at least one control element (also referred to herein as an intraluminal extension), such as a first control element 134. The first control element 134 may be connected anywhere to the first pump 130, projecting therefrom. For example, the first control element 134 may be connected to and may project from the first pump electrical connector 133 of the first pump 130. Alternatively, the first control element 134 may be connected to and may project from a corresponding pump physical connector (not shown; described hereinafter). The first control element 134 extends from the first pump 130 such that, when the modular pump system 100 is delivered or implanted, the first control element 134 may extend intraluminally, such as through the vasculature. As such, the proximal end portion of the first control element 134 may be disposed intraluminally (e.g., in the vasculature), extraluminally (e.g., outside the vasculature but still in a subject's body), and extracorporeally.

Projecting from the first pump 130 and slidably received in a corresponding control element passage guide of the pump dock 110, the first control element 134 is manipulable, at a proximal end portion thereof, by an operator to dock the first pump 130 to the pump dock 110 and to undock the first pump 130 from the pump dock 110.

More specifically, with the first pump 130 positioned distally relative to the pump dock 110, the proximal end portion of the first control element 134 may be pulled away relative to the pump dock 110 to dock the first pump 130 to the pump dock 110 and to connect the first pump electrical connector 133 to the to the first dock electrical connector 112a. Conversely, the proximal end portion of the first control element 134 may be pushed toward the pump dock 110 to undock the first pump 130 from the pump dock 110 and to disconnect the first pump electrical connector 133 from the first dock electrical connector 112a.

Alternatively, with the first pump 130 positioned proximally relative to the pump dock 110, the proximal end portion of the first control element 134 may be pushed toward the pump dock 110 to dock the first pump 130 to the pump dock 110 and to connect the first pump electrical connector 133 to the to the first dock electrical connector 112a. Conversely, the proximal end portion of the first control element 134 may be pulled toward the pump dock 110 to undock the first pump 130 from the pump dock 110 and to disconnect the first pump electrical connector 133 from the first dock electrical connector 112a.

When the modular pump system 100 is delivered and implanted in vivo, the docking and connection of the first pump 130 to the pump dock 110 as well as the undocking and disconnection of the first pump 130 from the pump dock 110 may be carried out intraluminally when the pump dock 110 is located in a lumen, such as in a subject's vasculature, and the first control element 134 extends therefrom along this lumen to position the proximal end portion thereof at an extraluminal or extracorporeal location to be manipulated by the operator.

The first control element 134 may be a guide wire or any other suitable wire, cable, and the like (in this case, also referred to herein as a first wire control element and a wire control element) that is configured to be manipulable at a proximal end portion thereof by an operator. Manipulation of the first control element 134 moves the corresponding first pump 130 relative to the pump dock 110 in order to dock and electrically connect the first pump 130 to the pump dock 110 for operation, and to undock and electrically disconnect the first pump 130 from the pump dock 110. The first wire control element 134 may be attachable or removably attachable to and detachable or removably detachable from the first pump 130 by manipulation by an operator. For example, the first wire control element 134 may be screwable to the first pump 130 so that the operator may rotate the first wire control element 134 to screw the first wire control element 134 to the first pump 130 and unscrew the first wire control element 134 from the first pump. The first control element 134 may also be a snare configured to capture and/or release the first pump 130. When attached, the first wire control element 134 may be used to pull and/or push the modular pump system 100, including the first pump 130.

Since the first pump 130 receives power via the pump dock 110 when electrically connected thereto, the first control element 134 is not required to be configured to conduct electricity for powering the first pump 130, as it is for example the case for the modular mammalian body implantable fluid flow influencing device described in International Patent Application Number PCT/ZA2020/050,022. The International Patent Application Number PCT/ZA2020/050,022 is incorporated herein by reference in its entirety, including all reference incorporated by reference therein, for all purpose as if fully set forth herein, except for any definition, subject matter disclaimer or disavowals, and except to the extent that the incorporated material is inconsistent with the express disclosure herein, in which case the language in the present disclosure controls.

Given that it is not configured to conduct electricity, the first control element 134 has a smaller cross-sectional size than if it would be required to conduct electricity, for example by integrating an electrical conductor material. Advantageously, this reduction in cross-sectional size enables the first control element 134 to have a reduced intraluminal cross-sectional footprint when routed thought the vasculature, and thus to have a reduced blood flow hinderance. The reduction in cross-sectional size and intraluminal cross-sectional footprint of the control element, is also advantageous when an implanted modular pump system 100 includes multiple pumps, each having a respective control element extending therefrom in a lumen of the vasculature. In this case, the multiple control elements may collectively cause blood flow hinderance, and providing power to the pumps via the pump dock enables a reduction in the overall intraluminal cross-sectional footprint of the control elements and associated blood hinderance thereof.

The first control element 134 may also be a catheter (in this case, also referred to herein as a first catheter control element and a catheter control element) configured to carry component(s) and/or fluid(s) to the first pump 130 and thus to optionally deliver same intraluminally. For example, a relatively stiff guide wire (also referred to herein as a stiff guide wire) may be routed in the first catheter control element to provide structural rigidity and or tactile feedback to the first catheter control element for improving intraluminal navigation of the first pump 130 (docked to or undocked from the pump dock 110), such as in the vasculature. For example, such modulable and improved structural rigidity and/or stiffness of the first catheter control element may be useful when the first pump 130 needs to be pushed toward the pump dock 110 by an operator, such as for docking and/or undocking purpose(s). Indeed, the improved structural rigidity and/or stiffness provided by the stiff guide wire to the first catheter control element may better transmit the pushing force to the pump dock 110 instead of being inappropriately bent or folded by such force to impede or prevent the manipulation intended by the operator.

Selection of wires of various stiffness and/or insertion and removal of such wires(s) at least partially in and out the first catheter control element may modulate the structural rigidity and/or stiffness of the first catheter control element and thus further improve intraluminal navigation and tactile feedback of the first pump 130.

The first catheter control element may also be used with a fluid flush device (not shown) to provide flush solution (e.g., dextrose flush) to the first pump 130. For example, the fluid flush device may provide a fluid at a high pressure (e.g., at a higher pressure than the intraluminal or intracavity pressure in which the device is implanted, such as >140 mm Hg in the arterial circulation) to the first pump 130 to prevent blood from entering the first motor 132. The fluid flush device may also provide fluid to the first pump 130 in a closed circuit to hydraulically entrain rotation of the first impeller 131 (e.g., in instance where the motor is not an electric motor).

As shown in FIG. 1B, the first pump 130 may include at least one pump electrical connector (also referred to herein as a first device electrical connector and a device electrical connector), such as the first pump electrical connector 133. The first pump electrical connector 133 is configured to electrically connect, either removably or not, to the corresponding first dock electrical connector 112a in such a way that the pump electrical connector 133 may terminate within, about, and/or adjacent to dock electrical connector 112a. When the first pump 130 is properly docked to the pump dock 110, the first dock electrical connector 112a delivers power to the first pump electrical connector for rotating the first impeller 131 of the first pump 130.

Hence, the first pump electrical connector 133 may not be configured only to receive electricity from the corresponding first dock electrical connector 112a when connected thereto, but also to dock the first pump 130 to the pump dock 110.

The first pump 130 may include multiple pump electrical connectors, such as two or three pump connectors, each of which being configured to operate at distinct phases.

The first pump 130 may include at least one pump physical connector (not shown in FIG. 1B). The pump physical connector, which does not receive electricity from the pump dock 110, is configured to cooperatively connect, either removably or not, to a corresponding dock physical connector of the pump dock 110. Similar to the dock physical connector, the pump physical connector may be: (i) configured to engage and/or maintain, in combination with the pump electrical connector, a physical docking interaction between the pump and the pump dock 110 (i.e., the physical connection component of the docking interaction between the pump and the pump dock 110 is shared by the pump physical connector and the pump electrical connector); or (ii) configured to engage and/or maintain, without the assistance of the pump electrical connector, a physical docking interaction between the pump and the pump dock 110 (i.e., the physical connection component and the electrical connection component of the docking interaction between the pump and the pump dock 110 is separated between the pump physical connector and the pump electrical connector, respectively). For example, the first pump 130 may include one pump electrical connector and one pump physical connector.

The first pump 130 may include one or more pump electrical connector(s) and/or one or more pump physical connector(s). For example, the first pump 130 may include at least one first pump electrical connector 133 configured for both the electrical powering of the first pump 130 via the pump dock 110 and the physical docking of the first pump 130 to the pump dock 110. Alternatively, the first pump 130 may also include at least one first pump electrical connector 133 configured for the electrical powering of the first pump 130 via the pump dock 110, and at least one pump physical connector configured for the physical docking of the first pump 130 to the pump dock 110.

When one or more pump electrical connector(s) and/or one or more pump physical connector(s) is/are provided to the first pump 130, a corresponding number of dock electrical connector(s) and/or dock physical connector(s), including a corresponding number of optional pump receiving surface(s), is/are provided to the pump dock 110.

The pump dock 110 and the first pump 130 are dockable together and connectable together to provide power to the first pump 130 for operation. More specifically, the pump dock 110 and the first pump 130 are dockable together and connectable together via (i) a cooperative inter-engagement between the dock electrical connector 112a and the first pump electrical connector 133; (ii) an optional cooperative inter-engagement between the dock physical connector and the first pump physical connector; and (iii) an optional cooperative inter-engagement mating between the pump receiving surface 111a and the first pump 130, such as on an outer face thereof.

Such cooperative inter-engagement allows for (i) a docking interaction between the pump dock 110 and the first pump 130 that is strong enough to at least reduce unwanted undocking and/or unwanted disconnecting of the first pump 130 from the pump dock 110 in operation; and (ii) providing an appropriate orientation of the first pump 130 (taken together or in combination with at least another pump, such as the second pump 140) to obtain a blood outflow that is sufficient for the intended medical purpose.

Although the pumps are described herein as being dockable and connectable to the pump dock as well as being undockable and disconnectable from the pump dock, it will be appreciated that the docking interaction between the pumps and the pump dock may be otherwise. For example, one or more pump(s) may be non-removably dockable and/or non-removably connectable to the pump dock.

As illustrated herein, the pumps docked to the pump dock are arranged in a parallel or one next to each other configuration in which the pumps are arranged longitudinally parallel relative to one another.

Alternatively, the pumps docked to the pump dock may be arranged in a flared configuration in which the distance separating the distal end portions of the pumps is greater than the distance separating the proximal end portions of the pumps.

Still alternatively, the pumps docked to the pump dock may be arranged in a reversed flared configuration in which the distance separating the proximal end portions of the pumps is greater than the distance separating the distal end portions of the pumps. The arrangement of the pumps as being in the parallel, flared, and reserved flared configurations depends on the design and integration, such their respective angles relative to the body of the pump dock, of the dock electrical connectors, pump electrical connectors, dock physical connectors, pump physical connectors, and pump receiving surfaces.

Although the modular pump system 100, as shown and described herein, includes a pump dock 110 as well as two pumps (i.e., FIGS. 1A to 1C) or three pumps (i.e., FIGS. 3A to 29C), the modular pump system 100 may be configured include any number of pumps.

The pumps described and illustrated herein include any implantable blood or heart pumps, such as axial-flow pumps, positive-displacement pumps, and centrifugal pumps.

Although the present disclosure refers to “pumps”, the skilled addressee will readily appreciate that the pumps may in fact be any other medical devices and equivalent structures that are capable of docking and connecting to the pump dock for operation or capable of undocking and disconnecting from the pump dock. This includes any medical devices and equivalent structures sized and shaped to be intraluminally delivered, intraluminally assembled or intraluminally docked to the pump dock, intraluminally operatively connected to the pump dock for operation, intraluminally implanted and/or intraluminally explanted. This also includes any medical devices and equivalent structures capable of slidable movement relative to the pump dock to dock thereto for operation, such as slidable movement inside a lumen.

Such other medical devices and equivalent structures include any blood flow influencing devices, balloon pump systems, intraluminal occluders, endovascular prosthesis, implantable pacemakers, interatrial shunt devices, valve replacement systems, and/or valve plasty systems.

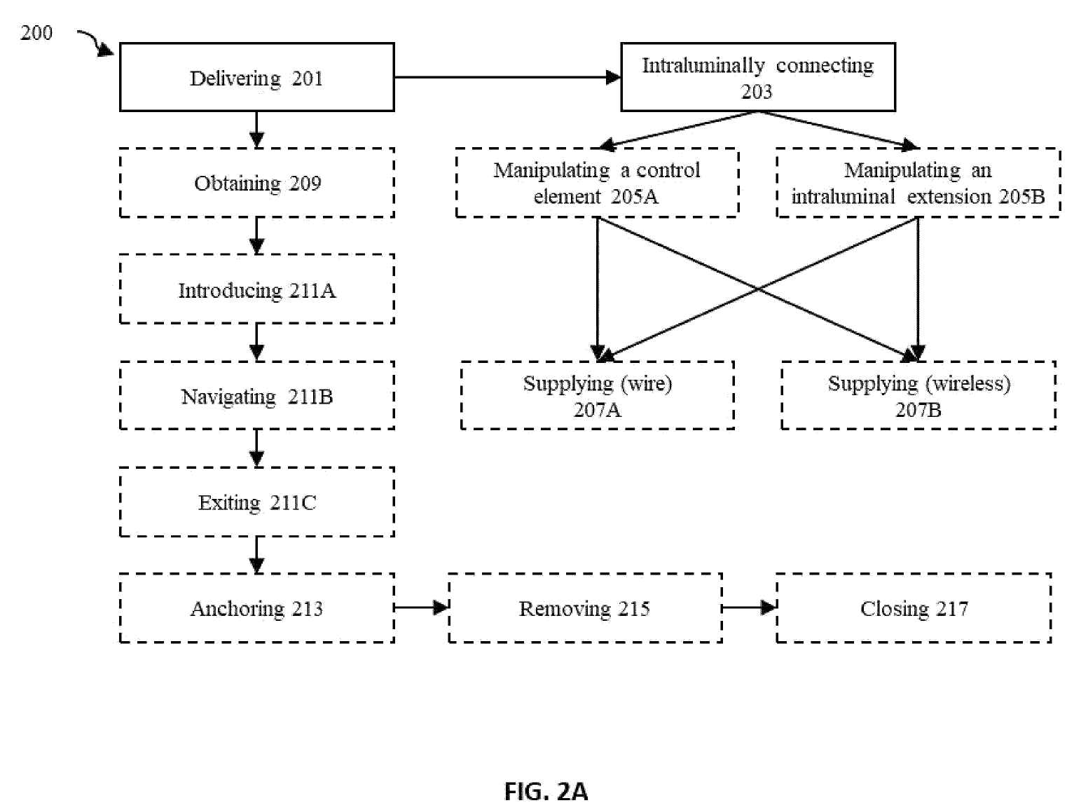

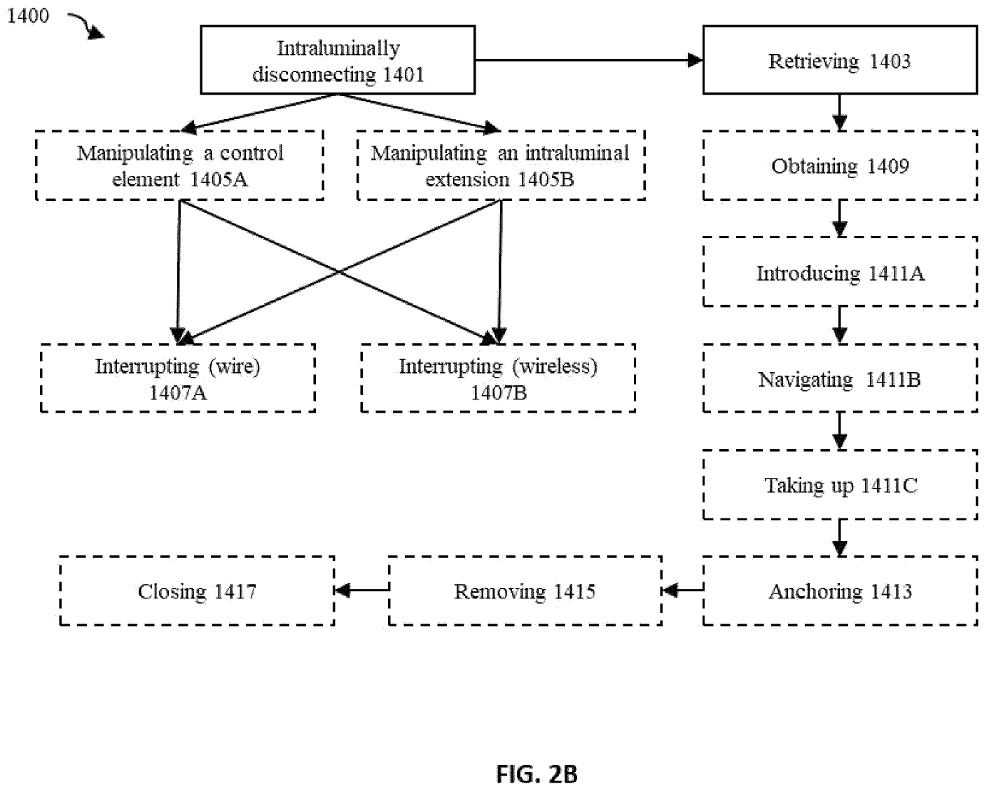

FIG. 2A shows a schematic flow diagram of a method 200 of implanting a modular pump system in a lumen, such as in the vasculature (including the lumen of the aorta and the chambers of the heart), a hollow organ, and a body cavity, according to an embodiment. In FIG. 2A, solid lines indicate elements of the method 200, and dotted lines indicate optional elements of method 200.

The method 200 of implanting a modular pump system (also referred to herein as an intraluminal modular powered medical system) to an intraluminal implantation site in a lumen of a subject includes: delivering the modular pump system to the intraluminal implantation site at 201, the modular pump system comprising a pump dock (also referred to herein as an intraluminal dock or an intraluminal control element guide) having a dock electrical connector and a pump (also referred to herein as an intraluminal medical device and an intraluminal powerable medical device) having a pump electrical connector (also referred to herein as a device electrical connector) that is electrically connectable to the dock electrical connector; and intraluminally connecting the dock electrical connector and the pump electrical connector together for powering the pump, at 203.

The intraluminally connecting 203 may include: manipulating a control element of the pump to intraluminally connect the pump electrical connector and the dock electrical connector together for powering the pump, at 205A. For example, the control element, at a proximal end portion thereof, may be positioned and manipulated intraluminally, intracorporeally (but not intraluminally), and/or extracorporeally. The control element may be in a slidable relationship with the pump dock, for example by being slidable therethrough, so that the control element is manipulated to slidably move the pump relative to the pump dock.

The connecting 203 may include: manipulating an intraluminal extension of the pump dock to intraluminally connect the dock electrical connector and the pump electrical connector together for powering the pump, at 205B. For example, the intraluminal extension, at a proximal end portion thereof, may be positioned and manipulated intraluminally, intracorporeally (but not intraluminally), and/or extracorporeally. The pump dock may be in a slidable relationship with the pump, such as with a control element provided thereto that is slidable through the pump dock, so that the intraluminal extension is manipulated to slidably move the pump dock relative to the pump.

The method 200 may further include: supplying power to the pump via the pump dock by electrically connecting, such as at a proximal end portion of a power source connector of the pump dock, the pump dock to a power source, at 207A; or by wirelessly transferring energy to the pump dock from a power source, at 207B.

The delivering 201 may include: obtaining an intraluminal access opening for delivering the modular pump system therethrough to the intraluminal implantation site, such as percutaneously, at 209.

The delivering 201 may further include: introducing a sheath or a catheter containing at least partially the modular pump system therein in the lumen through the intraluminal access opening, at 211A; intraluminally navigating or intraluminally guiding the sheath or catheter through the lumen up to the intraluminal implantation site thereof, at 211B, and exiting (or promoting the exit of) the modular pump assembly from the sheath or catheter in the lumen at the intraluminal implantation site thereof, at 211C.

The method 200 may further include: anchoring the modular pump system, such as via an anchor provided to the pump dock, at the intraluminal implantation site, at 213.

The delivering 201 may further include: removing a sheath or a catheter from the lumen after the modular pump system has been exited (or promoted to exit) therefrom at the intraluminal implantation site, at 215.

The delivering 201 may further include: closing the intraluminal access opening, such as by a chirurgical procedure, at 217.

Docking the pump to the pump dock may be performed by pulling the control element of the pump to move the pump toward the pump dock for docking and connecting the pump thereto (i.e., when the pump is positioned distally relative to the pump dock, with the pump electrical connector (also referred to herein as device electrical connector) being disposed proximally to the pump, and the dock electrical connector being disposed distally to the pump dock). Alternatively, the control element of the pump may be pushed to move the pump toward the pump dock for docking and connecting the pump thereto (i.e., when the pump is positioned proximally relative to the pump dock, with the pump electrical connector being disposed circumferentially to the pump, and the dock electrical connector being disposed circumferentially to the pump dock).

Docking the pump dock to the pump may also be performed by pushing an intraluminal extension, including a power source connector, of the pump dock (e.g., using a push rod abutting on the pump dock) to move the pump dock toward the pump for docking and connecting the pump dock to the pump (i.e., when the pump dock is positioned proximally relative to the pump, with the dock electrical connector being disposed distally to the pump dock, and the pump electrical connector (also referred to herein as device electrical connector) being disposed proximally to the pump). Alternatively, the power control element of the pump dock may be pulled to move the pump dock toward the pump for docking and connecting the pump dock to the pump (i.e., when the pump dock is positioned distally relative to the pump, with the dock electrical connector being disposed circumferentially to the pump, and the dock electrical connector being disposed circumferentially to the pump dock).

It will be appreciated that the control element of the pump and/or the power control element of the pump dock may be manipulated (e.g., by pulling and/or pushing a single one of them or both of them) to achieve docking between the pump and the pump dock.

In some embodiments where the modular pump system includes two pumps (i.e., first and second pumps provided with first and second control elements, respectively), the first and second pumps may be implanted from the intraluminal implantation by manipulating the first and second control elements, respectively, as per the method of implanting a modular pump system described herein, with the necessary change(s) appreciable to the skilled addressee having been made, if applicable.

FIG. 2B shows a schematic flow diagram of a method 1400 of explanting a modular pump system from a lumen, such as from the vasculature (including the lumen of the aorta and the chambers of the heart), a hollow organ, and a body cavity, according to an embodiment. In FIG. 2B, solid lines indicate elements of the method 1400, and dotted lines indicate optional elements of method 1400.

The method 1400 of explanting a modular pump system (also referred to herein as an intraluminal modular powered medical system) from an intraluminal implantation site in a lumen of a subject, the modular pump system comprising a pump dock (also referred to herein as an intraluminal dock or an intraluminal control element guide) having a dock electrical connector and a pump (also referred to herein as an intraluminal medical device and an intraluminal powerable medical device) having a pump electrical connector (also referred to herein as a device electrical connector) that is electrically disconnectable from the dock electrical connector, the method comprising: intraluminally disconnecting the dock electrical connector and the pump electrical connector from one another for unpowering the pump, at 1401; and retrieving the modular pump system from the intraluminal implantation site, at 1403.

The intraluminally disconnecting 1401 may include: manipulating a control element of the pump to intraluminally disconnect the pump electrical connector and the dock electrical connector from one another for unpowering the pump, at 1405A. For example, the control element, at a proximal end portion thereof, may be positioned and manipulated intraluminally, intracorporeally (but not intraluminally), and/or extracorporeally. The control element may be in a slidable relationship with the pump dock, for example by being slidable therethrough, so that the control element is manipulated to slidably move the pump relative to the pump dock.

The connecting 1401 may include: manipulating an intraluminal extension of the pump dock to intraluminally disconnect the dock electrical connector and the pump electrical connector from one another for unpowering the pump, at 1405B. For example, the intraluminal extension, at a proximal end portion thereof, may be positioned and manipulated intraluminally, intracorporeally (but not intraluminally), and/or extracorporeally. The pump dock may be in a slidable relationship with the pump, such as with a control element provided thereto that is slidable through the pump dock, so that the intraluminal extension is manipulated to slidably move the pump dock relative to the pump.

The method 1400 may further include: interrupting the supply of power to the pump via the pump dock by electrically disconnecting, such as at a proximal end portion of a power source connector of the pump dock, the pump dock from a power source, at 1407A; or by interrupting a wireless energy transfer from a power source to the pump dock, at 1407B.

The retrieving 1403 may include: obtaining an intraluminal access opening for retrieving the modular pump system therethrough from the intraluminal implantation site, such as percutaneously, at 1409.

The retrieving 1403 may further include: introducing a sheath or a catheter in the lumen through the intraluminal access opening, at 1411A; intraluminally navigating or intraluminally guiding the sheath or catheter through the lumen up to the intraluminal implantation site thereof, at 1411B, and taking up (or promoting the taking up of) at least partially the modular pump assembly in the sheath or catheter from the lumen at the intraluminal implantation site thereof, at 1411C.

The method 1400 may further include: unanchoring the modular pump system, such as via an anchor provided to the pump dock, from the intraluminal implantation site, at 1413.

The method 1400 may further include: removing a sheath or a catheter containing at least partially the modular pump system therein from the lumen, at 1415.

The method 1400 may further include: closing the intraluminal access opening, such as by a chirurgical procedure, at 1417.

Undocking the pump from the pump dock may be performed by pushing the control element of the pump to move the pump away from the pump dock for undocking and disconnecting the pump therefrom (i.e., when the pump is positioned distally relative to the pump dock, with the pump electrical connector (also referred to herein as device electrical connector) being disposed proximally to the pump, and the dock electrical connector being disposed distally to the pump dock). Alternatively, the control element of the pump may be pulled to move the pump away from the pump dock for undocking and disconnecting the pump therefrom (i.e., when the pump is positioned proximally relative to the pump dock, with the pump electrical connector being disposed circumferentially to the pump, and the dock electrical connector being disposed circumferentially to the pump dock).

Undocking the pump dock from the pump may also be performed by pulling the intraluminal extension, including a power source connector, of the pump dock to move the pump dock away from the pump for undocking and disconnecting the pump dock from the pump (i.e., when the pump dock is positioned proximally relative to the pump, with the dock electrical connector being disposed distally to the pump dock, and the pump electrical connector (also referred to herein as device electrical connector) being disposed proximally to the pump). Alternatively, the power control element of the pump dock may be pushed (e.g., using a push rod abutting on the pump dock) to move the pump dock away from the pump for undocking and disconnecting the pump dock from the pump (i.e., when the pump dock is positioned distally relative to the pump, with the dock electrical connector being disposed circumferentially to the pump, and the dock electrical connector being disposed circumferentially to the pump dock).

It will be appreciated that the control element of the pump and/or the power control element of the pump dock may be manipulated (e.g., by pulling and/or pushing a single one of them or both of them) to achieve undocking between the pump and the pump dock.

In some embodiments where the modular pump system includes two pumps (i.e., first and second pumps provided with first and second control elements, respectively), the first and second pumps may be explanted from the intraluminal implantation by manipulating the first and second control elements, respectively, as per the method of explanting a modular pump system described herein, with the necessary change(s) appreciable to the skilled addressee having been made, if applicable.

In some embodiments where the modular pump system includes more than one pumps, it will further be appreciated that each of the pump electrical connectors is connectable to and disconnectable from a corresponding connector of the dock electrical connectors (i) together simultaneously and (ii) in a stepwise manner relative to one another (e.g., by connecting or disconnecting a first pump, then a second pump, and so on), for example depending on how the operator manipulates the proximal end portions of the respective control elements, respectively. Accordingly, same holds true for the docking and undocking of the pump relative to the pump dock.

The intraluminal implantation site may include an implantation site located in a lumen of a body conduit, such as in a lumen of the vasculature, including the lumen of the aorta and the chambers of the heart, a hollow organ, and a body cavity.

The modular pump system is configured to be transcatheterally delivered for implantation at an intravascular implantation site and to be transcatheterally retrieved for explantation from the intravascular implantation. For example, the intravascular implantation site may be the lumen of the aorta or a chamber of the heart.

For implantation, in use, the modular pump system, which is contained in a catheter (or sheath), is delivered with at least one pumps undocked and thus disconnected from the pump dock (that is, in an undocked configuration). The catheter (or sheath) is routed through the vasculature up to or near to the intravascular implantation site. Then, the modular pump system is exited from the catheter (or sheath) at or close to the implantation site, and the pump is docked to the pump dock, resulting in a docked configuration.

For explantation, in use, the modular pump system in the docked configuration is retrieved from the intravascular implantation site by undocking and thus disconnecting at least one pump from the pump dock. Then, the modular pump system in the undocked configuration is taken up within a catheter (or sheath), and the catheter is removed from the vasculature.

While the implantation and the explanation of the modular pump system is described herein as being performed by a transcatheter procedure, either percutaneously or not, it will be appreciated that the modular pump system can also be implanted and explanted without a catheter (or sheath).

For implantation, in use, the modular pump system is routed through the vasculature up to the intravascular implantation site without being contained in a catheter (or sheath), and at least one pump is docked to the pump dock. The routing of the pump in the vasculature may be performed by pulling and/or pushing on at least one control elements and/or by snaring the pump, as known in the art.

For explanation, in use, at least one pump is undocked from the pump dock, and the modular pump system is removed from the vasculature without being contained in a catheter (or sheath). The retrieval of the pump may be performed by pulling on at least one control element and/or by snaring the pump.

FIGS. 3A-5C illustrate a modular pump system 300, according to an embodiment. Modular pump system 300 can be similar to or the same as, in form and/or function, modular pump system 100. Thus, portions and components of modular pump system 300 are not described in further detail herein. As shown in FIGS. 3A-3D, modular pump system 300 includes a first pump 330, a second pump 340, and a third pump 350, as well as a pump dock 310 to which the three pumps can docked. As shown in FIGS. 3A and 3D, specifically, pump dock 310 includes an anchor 315 configured to anchor or secure pump dock 310 (e.g., with pumps 330, 340, and 350 docked thereto) in a lumen of a subject's anatomy.

FIG. 3A illustrates pump 330 docked to pump dock 310, and pumps 340 and 350, operably coupled to but not yet docked to pump dock 310. FIG. 3B illustrates all three pumps 330, 340, 350 docked to pump dock. The pumps 330, 340, 350 has pump electrical connectors 333, 343, 353 (also referred to herein as device electrical connectors), respectively. Each pump electrical connectors 333, 343, 353 is a single prong with three conductive sections or rings CR. Each conductive sections or rings CR is suitable for a different phase of the motor (not shown) of modular pump system 300. Although in this embodiment each pump includes a single prong with three conductive sections or rings CR, in some embodiments, each pump 330, 340, 350 can include multiple prongs, e.g., three prongs, with each of the three prongs being suitable for a different phase of the motor (e.g., with each prong having one conductive section or ring).

FIG. 3C illustrates pump 330 (which can be the same as or similar to, in form and/or function, pumps 340 and 350, including pump electrical connector 133), and FIG. 3D shows the pump electrical connector 133 in more detail. As shown, pump electrical connector 133 is at a terminal end of pump 330, such that pump 330 can be advanced (e.g., pushed or pulled) towards and into contact with pump receiving surface 311a (when present) and dock electrical connector 312A.

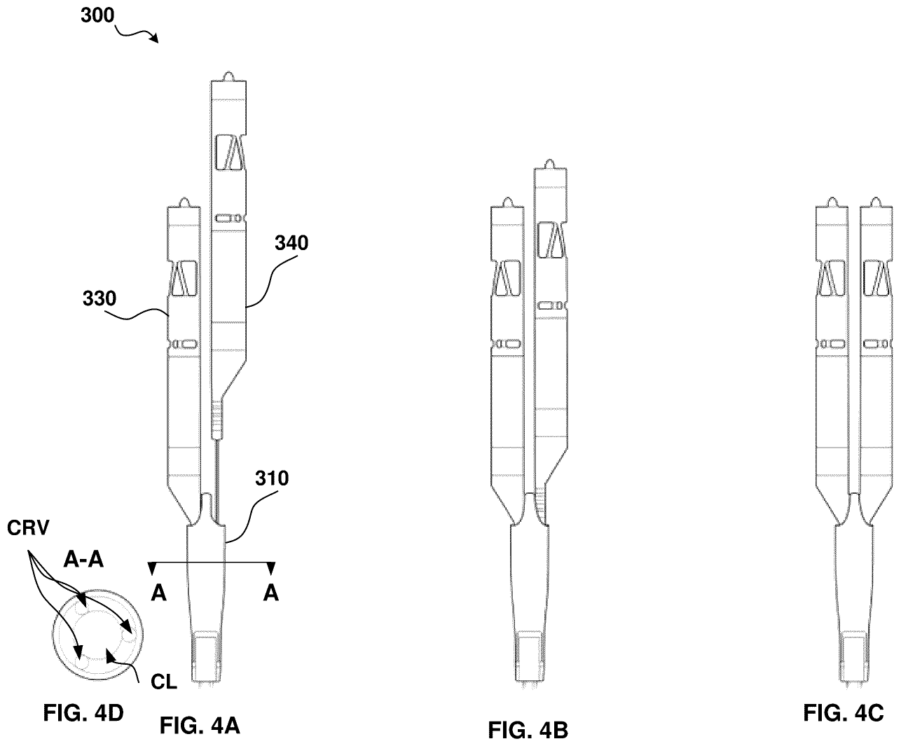

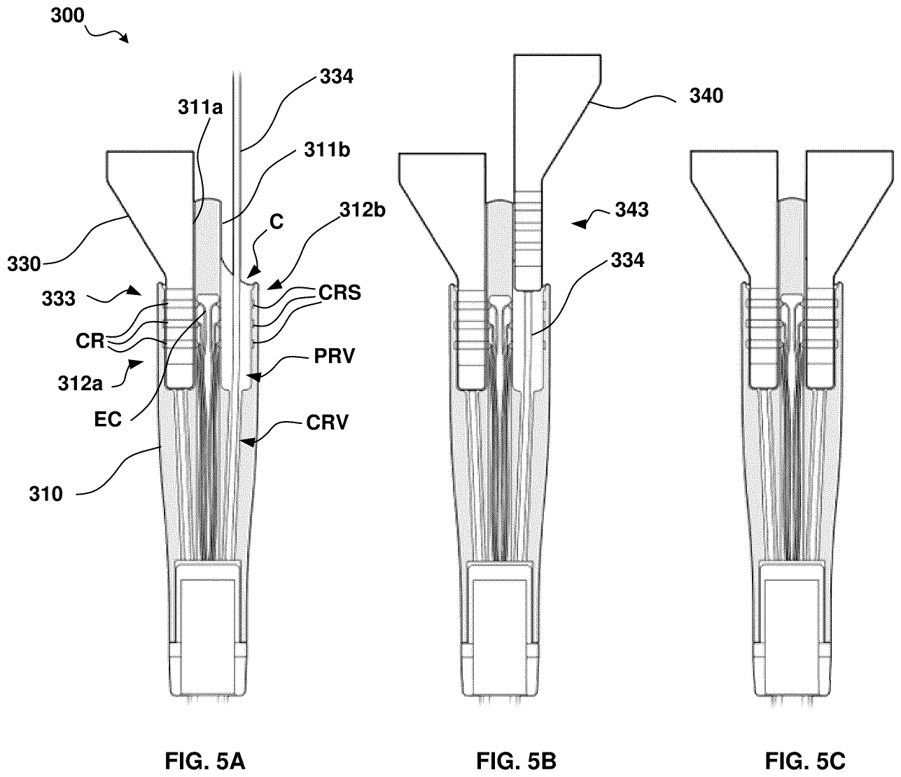

FIGS. 4A-C illustrate this process. In FIG. 4A, pump 330 is docked to pump dock 310, and pump 340 is in an undocked configuration, with its control element 344 routed through the dock electrical connector 312b, as shown and described in further detail with reference to FIGS. 5A-5C. FIG. 4B illustrates pump 340 in an intermediate configuration between an undocked configuration and a docked configuration. FIG. 4C illustrates pump 340 docked to pump dock 310. FIG. 4D illustrates a top view cross-section taken at A-A in FIG. 4A of dock 310. FIG. 4D shows lumens (also referred to herein as control element receiving volumes CRVs and control element passage guides) that are circumferentially distributed about a central axis of pump dock 310 and configured to receive control elements 334, 344, 354 for connecting and docking to each pump 330, 340, 350. When the pumps 330, 340, 350 are so docked and connected, the control elements 334, 344, 355 of pumps 330, 340, 350 extend along corresponding lumens through and beyond dock 310 to reach an operator for manipulation. Also, shown in FIG. 4D and FIGS. 5A-5C is an inner or central lumen CL through which the electrical connectors EC can be routed.

As shown, pump dock 310 defines pump receiving surface 311a, 311b, 311c for each of pumps 330, 340, 350, respectively, and three dock electrical connectors 312a, 312b, 312c (also referred to herein as dock electrical connectors) to receive the pump electrical connectors 333, 343, 353, respectively, and their respective control elements 334, 344, 354, as shown in relatively more detail in the docking sequence illustrated in FIGS. 5A-5C. As shown in FIGS. 4A-4C and 5A-5C, the pump receiving surface 311a, 311b, 311c are at least partially defined by a projection projecting distally in a direction opposite to the catheter 316. As shown, each dock electrical connector 312a, 312b, 312c define a cavity C configured to receive (and circumferentially surround) each pump electrical connector 333, 343, 353, respectively, including their respective control elements 334, 344, 354. Each cavity C defines a receiving volume RV for each prong of pump electrical connector 333, 343, 353 and their respective conductive sections or rings CR, including conductive receiving sections CRS, thereby collectively providing physical mating or securement of the components. The cavity C may include a fluid flush port (not shown) provided to the bottom thereof for evacuating the fluid contained in the cavity C and the receiving volume RV when a pump electrical connector is inserted therein for establishing an electrical connection.

In this embodiment, each conductive receiving section CRS includes a compressive conductive component, e.g., a spring, coil, and/or the like, that is configured to align with and circumferentially surround and establish sufficient electrical contact with its respective conductive ring CR when each pump electrical connector 333, 343, 353 is docked. For example, pump electrical connectors 333, 343, 353 (conductive sections or rings CR) and/or the dock electrical connector 312a, 312b, 312c may be spring-loaded to provide sufficient contacting force between the connectors to establish an electrical connection, even in presence of blood. In this manner, for example, each conductive coil may compensate for any misalignment, tolerance variations, and/or mating surface irregularities, and thereby provide electrical contact redundancy. Additionally, in some implementations, the compressive conductive component can contribute to coupling or securement of the pump electrical connector 333, 343, 353 within the cavity C by compressive force coupled to and extending from each conductive receiving section CRS are electrical connectors EC (e.g., wires, cables, and/or the like) configured to establish an electrical connection with their respective conductive ring CR, as illustrated in FIGS. 5A-5C. Each electrical connector EC is then routed towards and electrically coupled with a power source connector (not shown), similar to the power source connector 113 described above.

Each cavity C, in addition to defining a prong receiving volume PRV to receive each pump electrical connector 333, 343, 353 prong, defines a control element receiving volume CRV (also referred to herein as control element passage guides) configured to receive a control element 334, 344, 354, as shown. In this manner, each control element 334, 344, 354 can be routed through the dock, first entering through a cavity C (including the prong receiving volume PRV and the control element receiving volume CRV), and out an exit portion or aperture of pump dock 310. Physical coupling or securement of each pump 330, 340, 350 can be provided by the relative shape and/or volume of the cavity C relative to the prong of the pump (e.g., cavity C can be shaped and/or sized relative to each prong of each pump electrical connector 333, 343, 353 to provide an interference fit). Additionally, or alternatively, such physical coupling or securement can be provided by tension applied by each control element 334, 344, 354. By configuring pump 340 to be physically secured to pump dock 310 via pump electrical connector 343 and dock electrical connector 312a's collective mating arrangement, pump 340 can be physically secured to pump dock 310 without any additional securement or retainment mechanisms.

Although in this embodiment pump dock 310 includes a particular number and arrangement of cavities, volumes, lumens, etc., in other implementations a dock can have any suitable number and arrangement of cavities, volumes, lumens, etc., e.g., to ensure that the components accommodated therein have sufficient space to function without any complications or interruptions therebetween. For example, although pump dock 310 is provided with female-type electrical connectors and pump 330, 340, 350 are provided with respective male-type connector, pump dock 310 is provided with male-type electrical connectors and pump 330, 340, 350 are provided with respective female-type connector.

Modular pump system 300 may further includes catheter 316 coupled to pump dock 310 and configured to extend from pump dock 310 to, for example, an extravascular location and/or outside the subject's body. Catheter 316 is also configured to define at least one lumen to accommodate power source connector 313, control elements 334, 344, 355, and/or optional control component 314, similar to as described with respect to catheter 116.

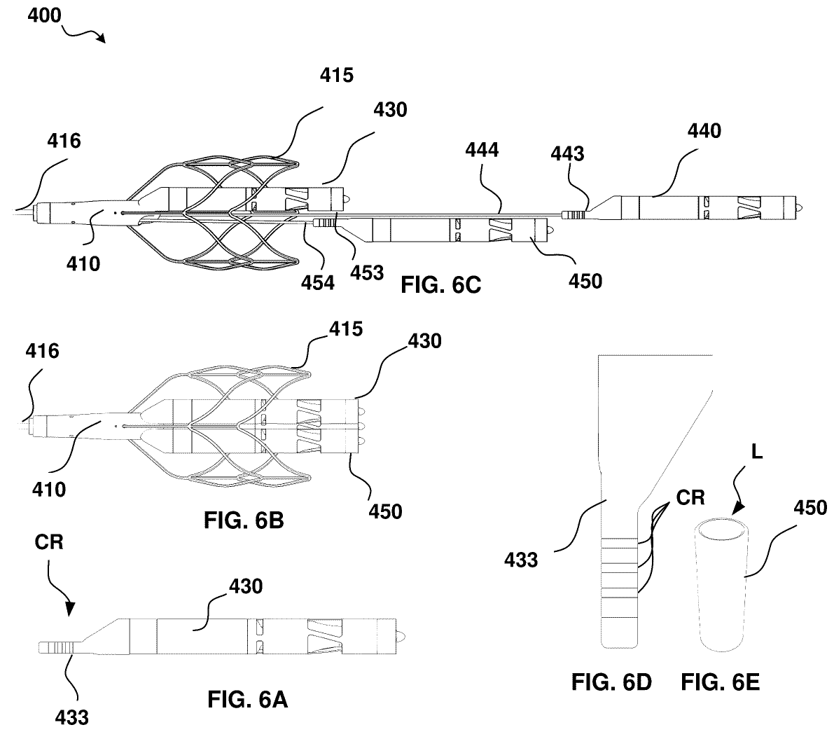

In some instances, it may be desirable to fluidically seal one or more cavities of a dock prior to docking of one or more pumps to the dock, e.g., to limit or prevent blood from entering the one or more cavities. FIGS. 6A-6E, 7A-D, and 8A-C illustrate a modular pump system 400 configured to provide such a fluidic seal, according to an embodiment. Modular pump system 400 can be similar to or the same as, in form and/or function, any of the modular pump systems described herein (e.g., modular pump system 100, modular pump system 300, etc.). Thus, portions of modular pump system 400 are not described in further detail herein, and rather, the focus will be on the portions of modular pump system 400 configured to provide the fluidic seal as described above.

As first shown in perspective view in FIG. 6E, and then in FIGS. 8A-C in cross-sectional side views, modular pump system 400 includes three seals 450 (also referred to herein as “plug”). Although in this embodiment there are three seals, in some embodiments, any suitable number of seals can be included, e.g., depending on and/or corresponding to the number of pumps within the modular pump system). Seals 450 can have any shape, size, and can be formed of any material, suitable to be inserted into and disposed within a cavity C of pump dock 410, and to plug or seal the respective cavity C to limit and/or prevent fluid (e.g., blood) from entering the cavity. In some implementations, for example, each seal 450 can be formed of silicon, thermoplastic elastomer, rubber, polytetrafluoroethylene, and/or the like. Each seal 450 defines a lumen L spanning its length such that a control element 434, 444, 454 can be routed therethrough. The lumen L can be shaped and sized so as to limit and/or prevent any fluid from traveling into or through the lumen L when a control element 434, 444, 454 is disposed therein. For example, lumen L can be just slightly larger in diameter than control elements 434, 444, 454 so that control elements 434, 444, 454 can translate therethrough. Further, in this manner, such an arrangement may limit or prevent control elements 434, 444, 454 from any undesirable twisting, kinking, or the like.

In this embodiment, each cavity C of the dock electrical connectors 412a, 412b, 412c (also referred to herein as dock electrical connectors) are shaped and sized to accommodate both (i) a pump electrical connector 433, 443, 453 (also referred to herein as device electrical connectors) (e.g., their respective prongs) from each pump 430, 440, 450 (similar to modular pump system 300), and (ii) one of the seals 450. As show in FIGS. 4A-4C and 5A-5C, the dock electrical connectors 412a, 412b, 412c are associated or integrated with pump receiving surfaces that are at least partially defined by a projection projecting distally in a direction opposite to the catheter 416.

For simplicity, the following description will focus on a single pump (i.e., pump 430) and associated components; but it should be appreciated that such description can equally apply to the other pumps (i.e., pump 440 and 450) and associated components of modular pump system 400. In use, as illustrated sequentially across FIGS. 8A-8C, pump dock 410 can be delivered to through the subject's vasculature with a seal 450 inserted in the cavity C, with one end the seal 450 terminated at the entrance to the cavity C, to limit and/or prevent blood (and/or other fluids) from entering the cavity C, as shown in FIG. 8A. Also, as shown, control element 434 is routed through the lumen L of seal 450.

When the operator is ready to dock pump 430 with pump dock 410, similar to as described in other embodiments herein, the operator can manipulate (e.g., push or pull) control element 434 to advance pump 430 into the cavity C. In this embodiment, however, the pump electrical connector 433 contacts seal 450, and thereby forces and/or drives seal 450 deeper into the cavity C such that seal 450 is seated at or near an end of the cavity C and/or at or near the interface of the prong receiving volume PRV and the control element receiving volume CRVs (also referred to herein as control element passage guides), as shown in FIG. 8C. In this manner, the cavity C can remain free (or substantially free) from blood (and/or other fluids) during delivery of pump dock 410 (via seal 450), during docking (collectively via pump electrical connector 433 and seal 450, and after docking (via pump electrical connector 433).

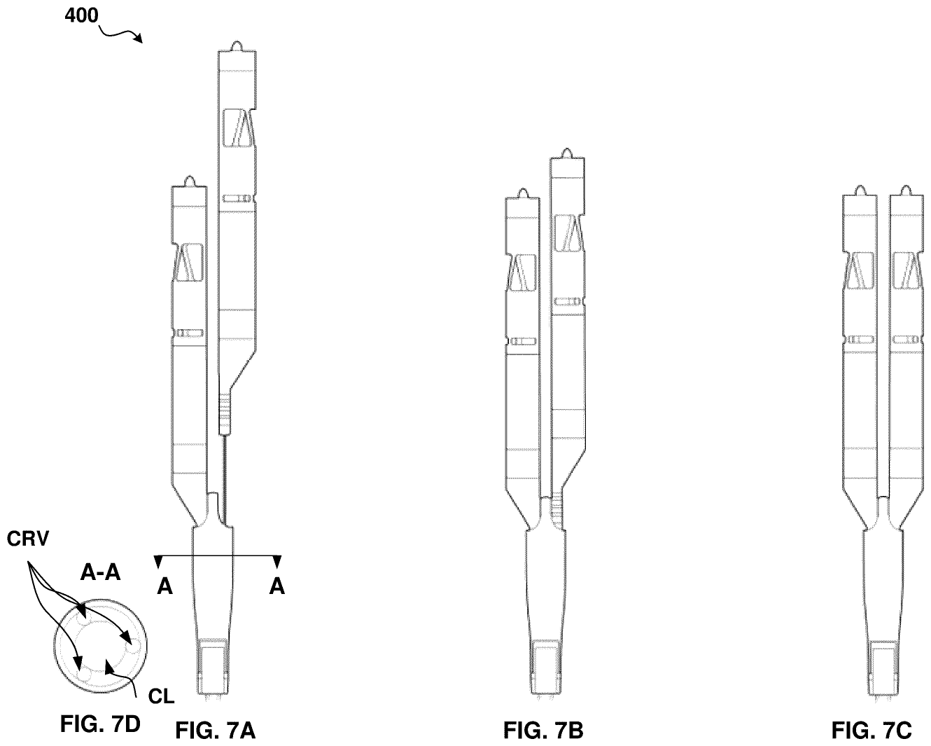

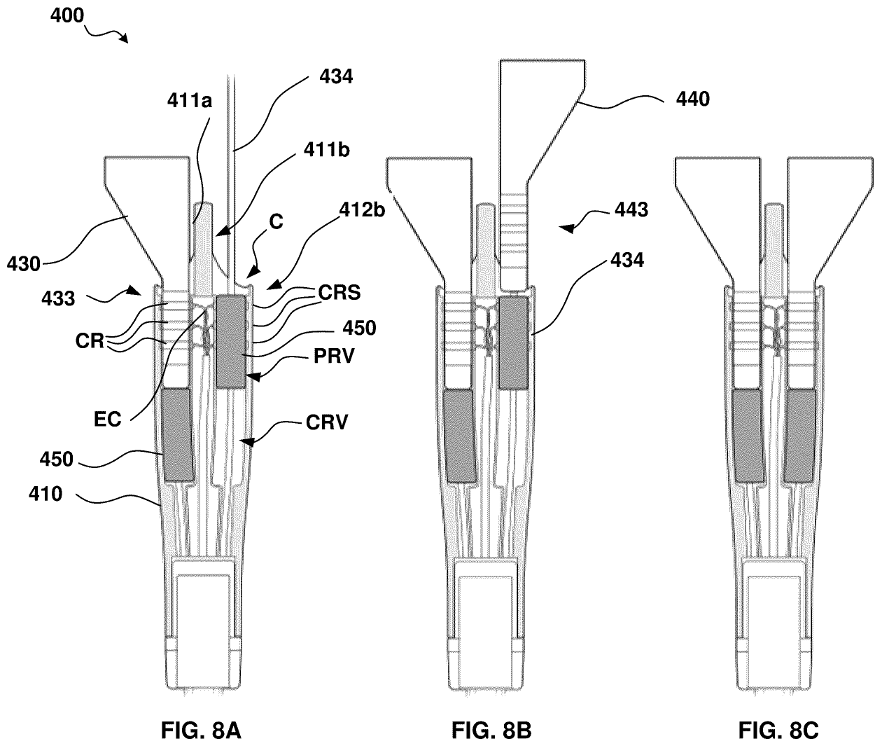

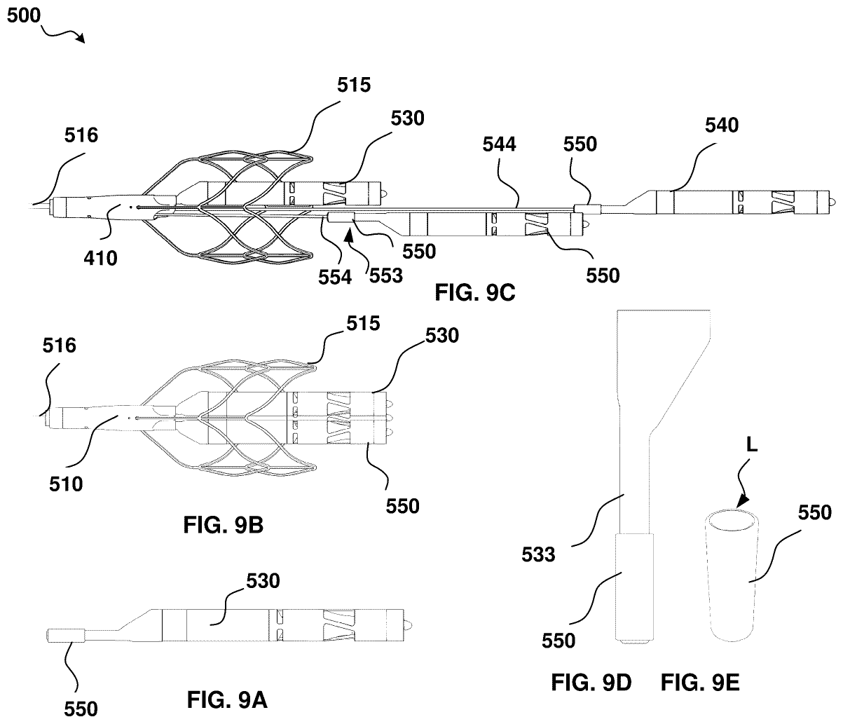

Although modular pump system 400 is shown and described as having three seals (i.e., one for each pump and each dock cavity), in some embodiments, more than three seals may be incorporated. FIGS. 9A-9E, 10A-10D, and 11A-11C illustrate a modular pump system 500 having six seals 550, according to an embodiment. Modular pump system 500 can be similar to or the same as, in form and/or function, modular pump system 400, except that modular pump system 500 includes additional seals 550, as described in further detail below. Thus, portions of modular pump system 500 are not described in further detail herein, and rather, the focus will be on the portions of modular pump system 500 configured to provide the fluidic seal via the six seals 550.

In this embodiment, three of the seals 550 are each configured to be coupled to and disposed about at least a portion of a pump electrical connector 533, 543, 553, as shown, for example, in FIGS. 9C, 9D, and 9E. These three seals will be referred to herein as 550A. Seals 550A are open at both ends, with a lumen L running therebetween to accommodate a pump electrical connector 533, 543, 553. The seals 550A are sized and shaped to fit about a pump electrical connector 533, 543, 553 with a snug fit (e.g., interference fit) thereby providing a fluidic seal therebetween when coupled. Further, at least a distal portion of each seal 550A is configured to have a cross-sectional area greater than the cross-sectional area of at least the entrance to the cavity C such that the distal portion of the seal 550A does not fit into the cavity C during docking, and instead abuts the entrance of the cavity C to fluidically seal the entrance during docking, as described in further detail below.

The other three seals 550 will be referred to herein as 550B, and can be the same as or similar to, in form and/or function, seals 450. All of the seals 550 can be formed of any suitable material. In some implementations, for example, the seals 550 are formed of silicon.

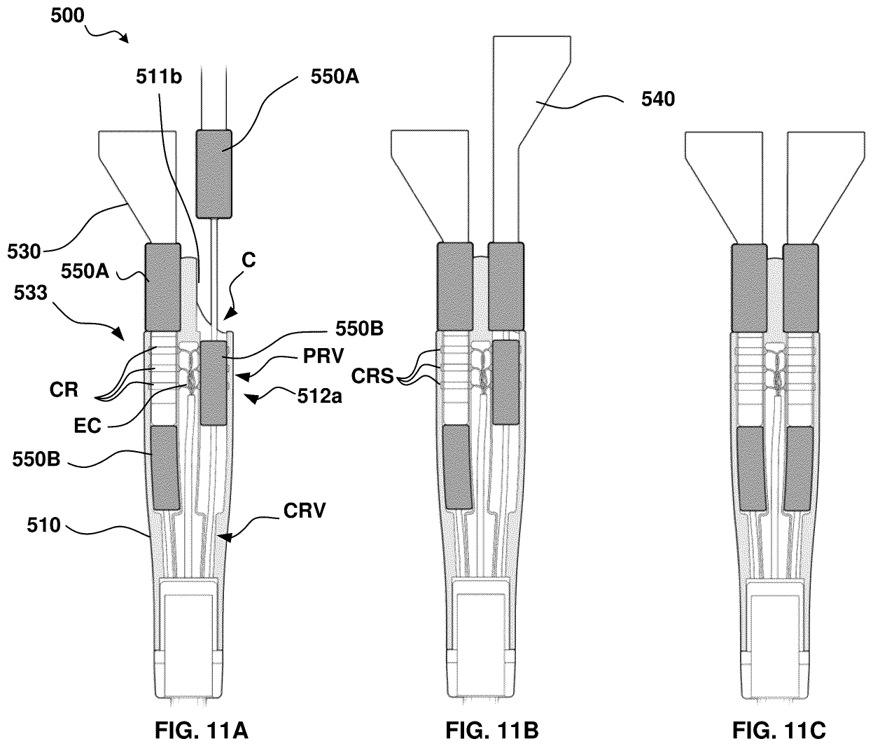

For simplicity, the following description will focus on a single pump (i.e., pump 530) and associated components; but it should be appreciated that such description can equally apply to the other pumps (i.e., pump 540 and 550) and associated components of modular pump system 500. In use, as illustrated sequentially across FIGS. 11A-11C, pump dock 510 can be delivered through a subject's vasculature with a seal 550B inserted in the cavity C, with one end the seal 550A terminating at the entrance to the cavity C, to limit and/or prevent blood (and/or other fluids) from entering the cavity C, as shown in FIG. 11A. Also, as shown, control element 534 is routed through the lumen L of seal 550B, according to an embodiment.

Further, pump 530 can be delivered through the subject's vasculature with a seal 550A coupled to and disposed about its pump electrical connector 533 (also referred to herein as device electrical connectors), as shown in FIG. 11A. When the operator is ready to dock pump 530 with pump dock 510, similar to as described in other embodiments herein, the operator can manipulate (e.g., push or pull) control element 534 to advance pump 530 into the cavity C. In this embodiment, however, the pump electrical connector 533 contacts seal 550B, and thereby forces and/or drives seal 550B deeper into the cavity C such that seal 550B is seated at or near an end of the cavity C and/or at or near the interface of the prong receiving volume PRV and the control element receiving volume CRVs (also referred to herein as control element passage guides), as shown in FIG. 11C. Further, as pump electrical connector 533 approaches the entrance to the cavity C for docking, seal 550A disposed about pump electrical connector 533, and in particular the distal portion of seal 550A, contacts both the pump receiving surface 511A and the portion of pump dock 510 that forms or defines the entrance to cavity C, thereby fluidically sealing cavity C, as shown in FIG. 11B. With seal 550A so sealed against pump dock 510, the operator can continue advancing pump 530 into the cavity C such that pump electrical connector 533 can advance distally relative to and through the lumen of the seal 550B, leaving the seal 550B in fluidic sealing contact with the entrance to the cavity C. In this manner, pump electrical connector 533 can be inserted into the cavity C for docking and to urge the seal 550A further into the cavity C, while maintaining a fluidic seal at the entrance to the cavity C via seal 550A.

As show in FIGS. 11A-11C, the dock electrical connectors are associated or integrated with pump receiving surfaces that are at least partially defined by a projection projecting distally in a direction opposite to the catheter.

Hence, the cavity C can remain free (or substantially free) from blood (and/or other fluids) during delivery of pump dock 510 (via seal 550A), during docking (collectively via seal 550B and pump electrical connector 533), and after docking (collectively via seal 550B and pump electrical connector 533).

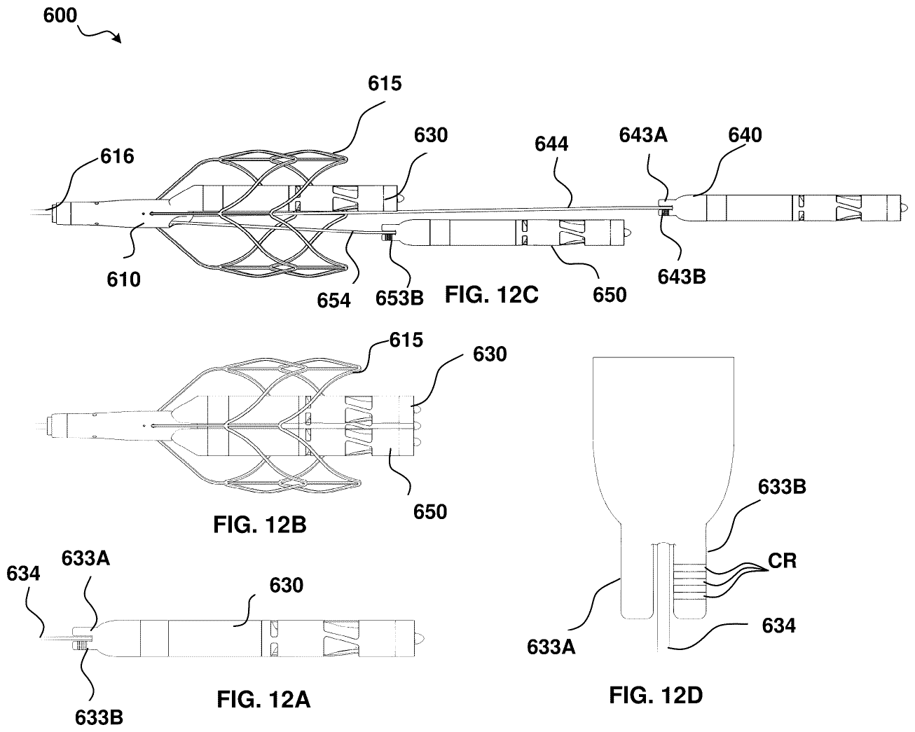

Although some embodiments described herein include pumps with a single prong to accommodate docking, in some instances, pumps can have more than one prong. FIGS. 12A-12D, 13A-13D, and 14A-14C illustrate such a modular pump system 600, according to an embodiment. Modular pump system 600 can be similar to or the same as, in form and/or function, any of the modular pump systems described herein, except that modular pump system 600 includes pumps 630, 640, 650, each having two prongs, a first being a physical connector 633A, 643A, 653A (also referred to as a pump physical connector or a device physical connector) to facilitate docking to pump dock 610, and a second being a pump electrical connector 633, 643, 653 (also referred to as a device electrical connector), being similar in form and function to other electrical connectors described herein (e.g., pump electrical connectors 333, 343, 353). Thus, portions of modular pump system 600 are not described in further detail herein.

For simplicity, the following description will focus on a single pump (i.e., pump 630) and associated components; but it should be appreciated that such description can equally apply to the other pumps (i.e., pump 640 and 650) and associated components of modular pump system 600. As shown, pump dock 610 defines two cavities: a physical connector cavity Cp (also referred to herein as a dock physical connector) and an electrical connector cavity Ce (also referred to herein as a dock electrical connector), being similar in form and function to other electrical connectors described herein (e.g., dock electrical connectors 312a, 312b, 312c).

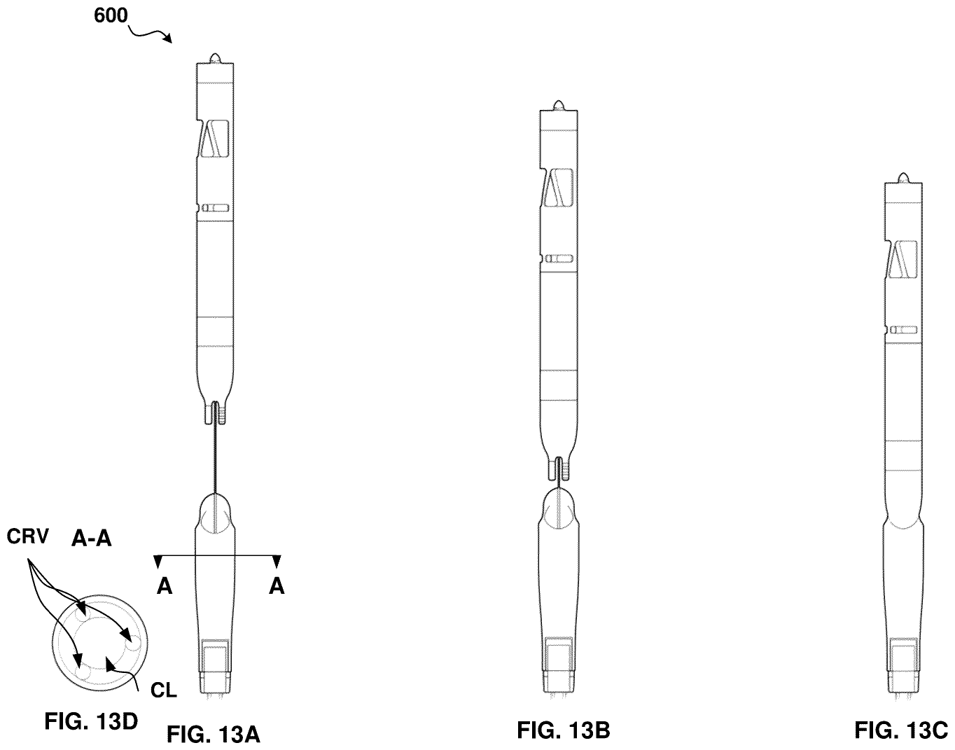

Physical connector cavity Cp is configured to receive and be secured to physical connector 633A when pump 630 is docked to pump dock 610, similar to as described in other embodiments herein. Electrical connector cavity Ce is similarly configured to receive and be secured to electrical connector 633B when pump 630 is docked to pump dock 610. Electrical connector cavity Ce defines a receiving volume RV for electrical connector 633B and its conductive sections or rings CR. Coupled to and extending from the electrical connector cavity Ce are electrical connectors EC (e.g., wires, cables, and/or the like) relatively spaced similar to the spacing of the conductive rings CR, and are configured to establish an electrical connection with their respective conductive ring CR, as illustrated in FIG. 13C. Each electrical connector EC is then routed towards and electrically coupled with a power source connector (not shown), similar to the power source connector 113 described above. Also, as shown, pump 630 includes a control element 634 extending from pump 630 between physical connector 633A and electrical connector 633B. Such an arrangement, in use, can help limit or prevent undesirable rotation of pump 630 relative to its axis (e.g., its central axis). Further, in some implementations, physical connector 633A and electrical connector 633B may have dissimilar shape, size, and/or material. For example, physical connector 633A may include metal, such as Nitinol, and/or may be shaped and/or sized to have a relatively tighter and/or more secure fit within physical connector cavity Cp.

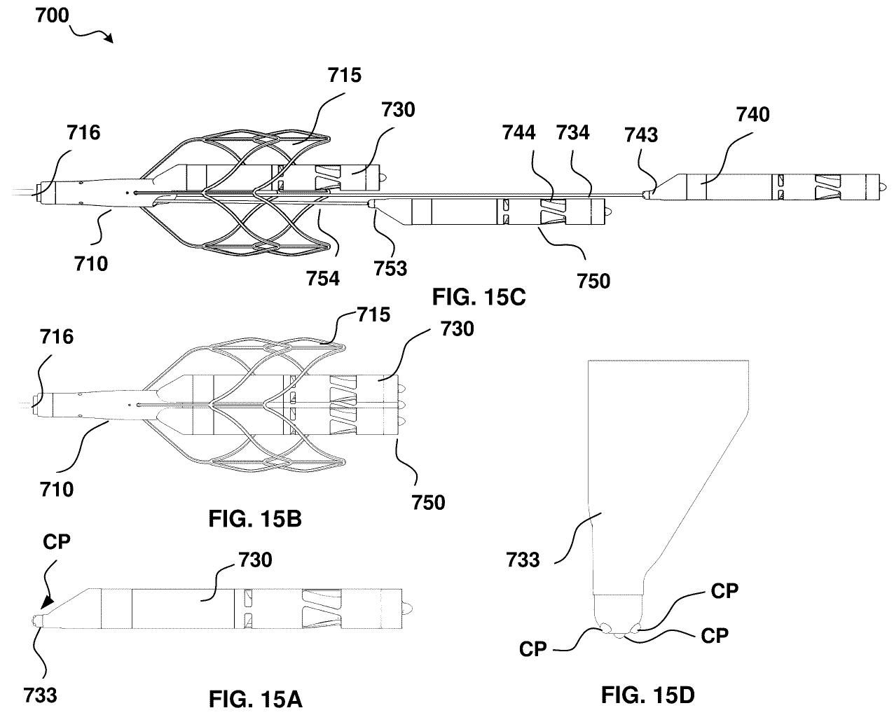

In some embodiments, rather than a pump having prongs and conductive rings as described in various embodiment herein, a pump may include a pump electrical connector having a prong of a round or otherwise curved surface. This prong may have conductive detents or protrusions extending therefrom that are configured to physically and electrically mate with electrical receivers disposed within a dock. FIGS. 15A-17C illustrate such a modular pump system 700, according to an embodiment. Modular pump system 700 can be similar to or the same as, in form and/or function, any of the modular pump systems described herein, except that modular pump system 700 includes pumps 730, 740, 750, each having a pump electrical connector 733, 743, 753 (also referred to herein as device electrical connectors), defining a curved distal end portion with three conductive protrusions CP (each configured to a separate phase, similar to as described with respect to conductive rings in various embodiments herein). Thus, portions of modular pump system 700 are not described in further detail herein.

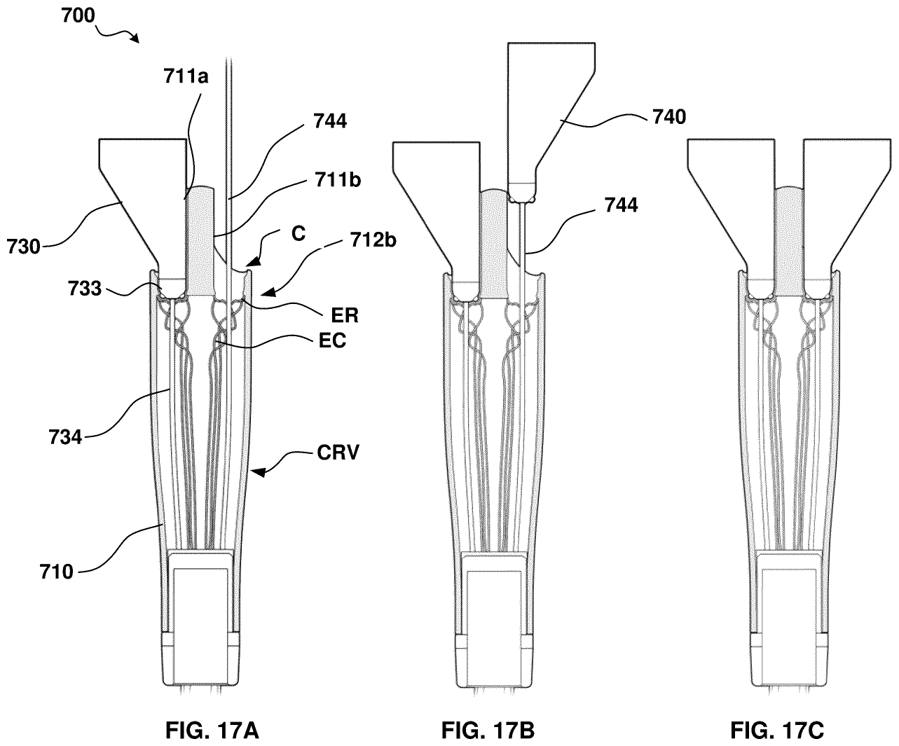

For simplicity, the following description will focus on a single pump (i.e., pump 730) and associated components; but it should be appreciated that such description can equally apply to the other pumps (i.e., pump 740 and 750) and associated components of modular pump system 700. As shown in FIGS. 17A-17C, pump dock 710 includes a dock electrical connector 712a (also referred to herein as dock electrical connector) having a cavity C and electrical receiver ER having three cups or curved shape portions configured to receive and/or mate with the conductive protrusions CP. The cavity C may include a fluid flush port (not shown) provided to the bottom thereof for evacuating the fluid contained in the cavity C and the receiving volume RV when a pump electrical connector is inserted therein for establishing an electrical connection. The conductive protrusions can have any suitable shape (e.g., a convex, dome, etc.) to mate with the electrical receiver ER. In some implementations, the conduct protrusions CP can allow for relatively larger exertion of force during docking to displace any undesirable fluid (e.g., blood) between each conductive protrusions CP and electrical receiver ER, thereby improving the electrical connection therebetween. Similar to some embodiments described herein, dock electrical connector 712a and pump electrical connector 733 can collectively be shaped and sized to form an interference fit when docked. Although in this embodiment pump dock 710 houses control element 734 and dock electrical connector 712a in the same lumen or volume, in some embodiments, pump dock 710 can include any suitable distinctive lumens, for example, a dock may include a first lumen for control element 734 and a second, distinct, lumen for dock electrical connector 712a (e.g., and its connection to power source connector 713).

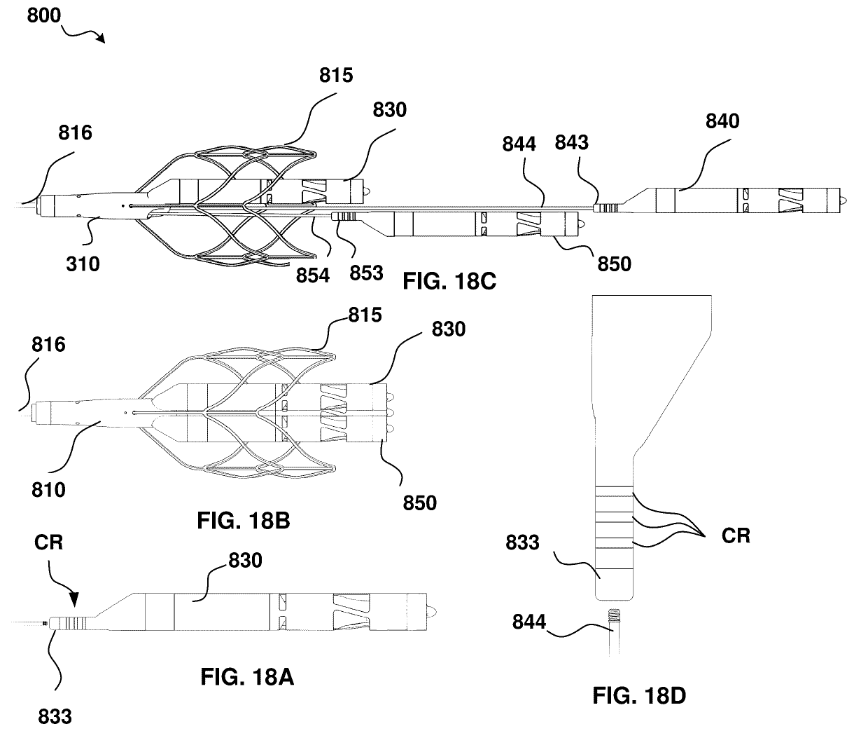

Various embodiments described herein include modular pump systems having pumps with control elements attached thereto and extending therefrom, to accommodate delivery and docking of those pumps. In some instances, it may be desirable to decouple a control element from a pump, e.g., after delivery and docking of that pump. FIGS. 18A-20C illustrate such a modular pump system 800, according to an embodiment. Modular pump system 800 can be similar to or the same as, in form and/or function, any of the modular pump systems described herein. Thus, portions of modular pump system 800 are not described in further detail herein.

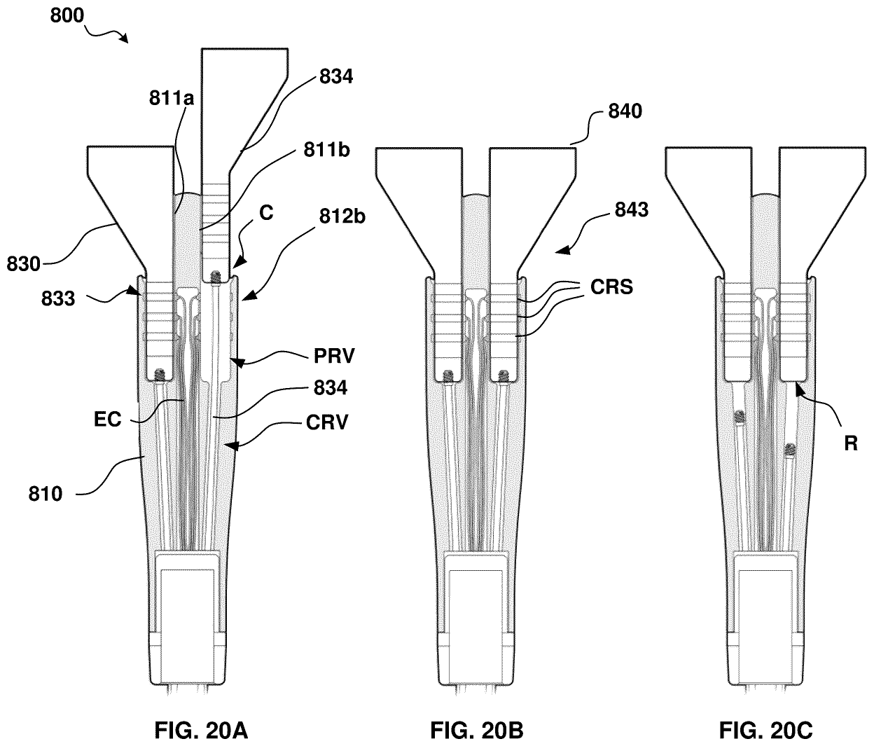

For simplicity, the following description will focus on a single pump (i.e., pump 830) and associated components; but it should be appreciated that such description can equally apply to the other pumps (i.e., pump 840 and 850) and associated components of modular pump system 800. In this embodiment, pump 830 is removably attachable to control element 834, and more specifically, pump 830 defines a threaded recess R configured to be threadedly coupled to a threaded coupling of control element 834, as shown in FIG. 20A. Threaded recess R is located at a distal end of the electrical connector 833 (also referred to herein as device electrical connector) of pump 830. Although in this embodiment threaded couplings are used, it should be noted that in some embodiments, any suitable coupling mechanism and/or arrangement can be used such that an operator can manipulate (e.g., pull, push, twist, etc.) the control element (e.g., from outside the subject) to decouple the control element from the pump. For example, the pump 830 may be removably attachable to control element 834 by a wire routed in the control element 834, the wire forming a loop or a lasso for removably attaching the pump 830 (i.e., just like a snare).

In use, as illustrated sequentially across FIGS. 20A-20C, with pump 830 coupled to control element 834, similar to previous embodiments described herein, pump 830 can be delivered through the subject's vasculature, and the operator can manipulate (e.g., push or pull) control element 834 to advance pump 830 into the cavity C defined by pump dock 810. Once docked, as shown in FIG. 20B, the operator can manipulate (e.g., twist, rotate, and the like) control element 834 to decouple and separate control element 834 from the threaded recess R and pump 830, and the operator can then withdraw control element 834 from pump dock 810 and, if desired, from the subject's vasculature or body.

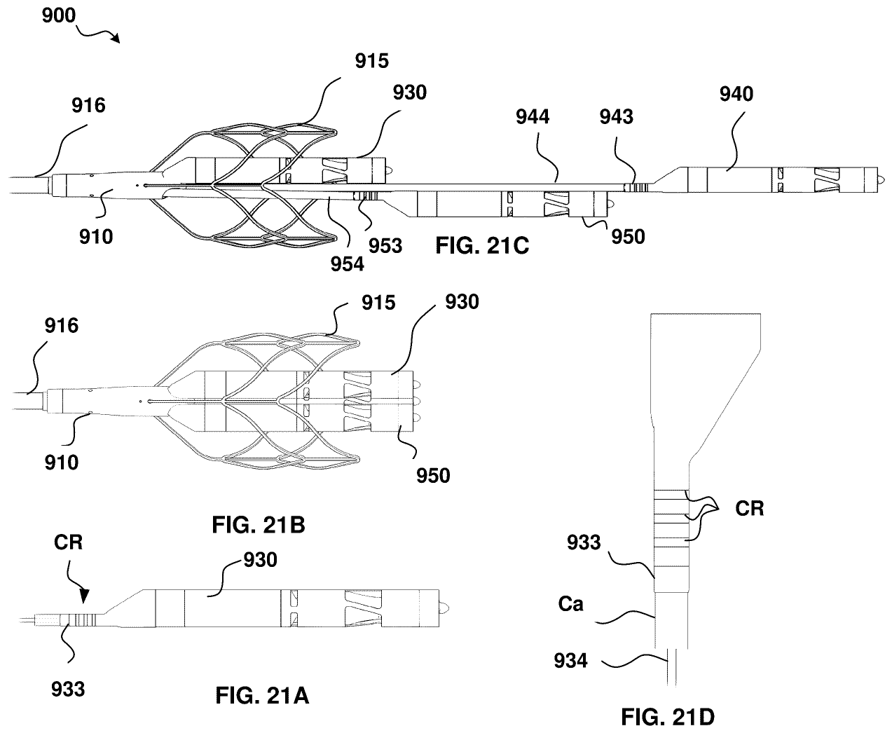

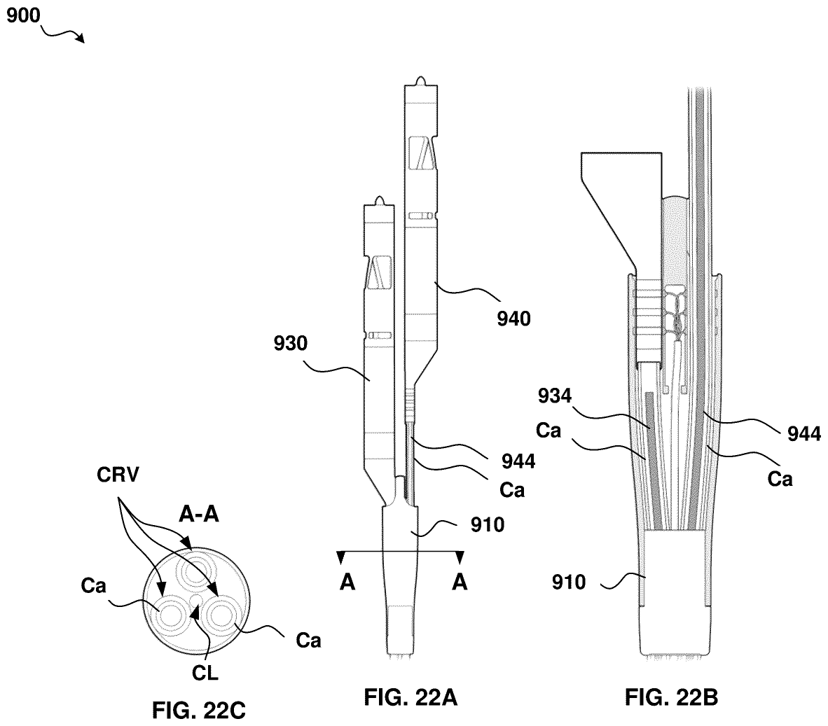

In some instances, it may be desirable to route the removably attachable control element through a catheter, whereby the control element is relatively more rigid/stiffer than the catheter, and provides sufficient rigidity and/or tactile feedback for the operator to manipulate the pump within the subject, but can decoupled and removed from the subject after docking, leaving the relatively more flexible catheter coupled to the pump. FIGS. 21A-22C illustrate such a modular pump system 900, according to an embodiment.

Modular pump system 900 can be similar to or the same as, in form and/or function, any of the modular pump systems (e.g., modular pump system 800) described herein. Thus, portions of modular pump system 900 are not described in further detail herein.

For simplicity, the following description will focus on a single pump (i.e., pump 930) and associated components; but it should be appreciated that such description can equally apply to the other pumps (i.e., pump 940 and 950) and associated components of modular pump system 900. Similar to modular pump system 800, in this embodiment, control element 934 is configured to be decoupled from pump 930, however here, control element 934 is routed through a lumen of a catheter Ca that is attached to and configured to extend from pump 930 through the subject's vasculature and outside the subject when the pump is docked and disposed at its implantation site. In this manner, the operator can manipulate control element 934 when it is coupled to pump 930 to dock pump 930 to pump dock 910, and then the operator can decouple and remove control element 934 from pump 930 (e.g., and from the subject's vasculature and/or body), leaving the catheter Ca coupled to and extending from docked pump 830. Accordingly, the relatively more rigid/stiffer control element 934 can be removed from the subject, while the relatively more flexible catheter can remain within the subject and provide access for introduction of additional components and/or fluids to the implantation site. Such flexibility is suitable to comply with the subject's vasculature.

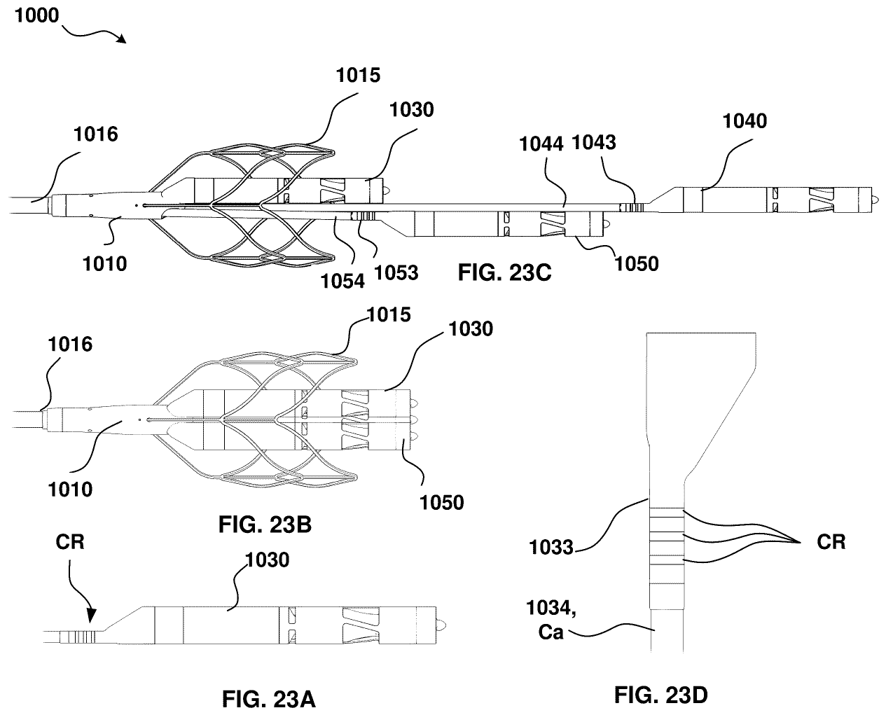

As discussed in connection with modular pump system 100, in some instances it may be desirable to flush a modular pump system to limit and/or prevent blood in a pump motor. In some instances, heat generated by a modular pump system may cook or otherwise cause blood to solidify or coagulate, e.g., into a jelly or gelatin consistency (i.e., causing the formation of a blood clot), which can jam or otherwise interrupt motor functionality. In some implementations, a pump has motor that is fully enclosed and has a magnetic coupling between the motor and impeller, and such cooked blood may find its way between those magnetically coupled parts, and so in those instances it would be beneficial to deliver a fluid to flush the pump and/or motor, and e.g., exit at the interface of the impeller and pump housing. Any suitable fluid can be used, such as, for example, dextrose (e.g., 5% or the like) or any other biocompatible fluid. FIGS. 23A-25D illustrate such a modular pump system 1000, according to an embodiment.

Modular pump system 1000 can be similar to or the same as, in form and/or function, any of the modular pump systems (e.g., modular pump system 900) described herein. Thus, portions of modular pump system 1000 are not described in further detail herein.

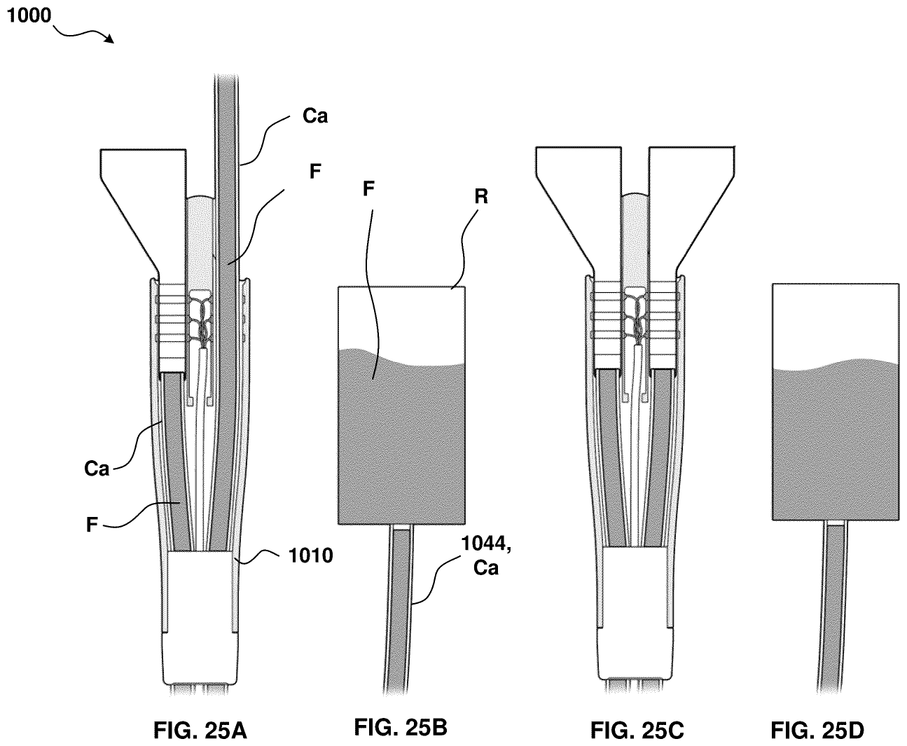

For simplicity, the following description will focus on a single pump (i.e., pump 1030) and associated components; but it should be appreciated that such description can equally apply to the other pumps (i.e., pump 1040 and 1050) and associated components of modular pump system 1000. Similar to modular pump system 900, in this embodiment, a catheter Ca is coupled to and extends from pump 1030 and is routed through pump dock 1010, as described in previous embodiments. The catheter Ca defines a lumen through which fluid F can be delivered from outside the subject and to pump 1030. The fluid F can be delivered from a reservoir R disposed outside the subject, for example, as shown in FIGS. 25B and 25D. In this embodiment the catheter Ca can serve both as a conduit to deliver the fluid, and as a control element 1034 for the operator to manipulate delivery, dock, and/or undock pump 1030, as described in further detail herein in connection with other embodiments. That said, in some embodiments, similar to as described with respect to modular pump system 900, the catheter can define a lumen through which a relatively more rigid (and detachable) control element can be routed, as well as a conduit for a fluid. In such embodiments, for example, the control element can be routed through the catheter and removably attached to a pump to facilitate docking of the pump, and then the control element can be detached from the pump and withdrawn through the catheter lumen, leaving the catheter coupled to the pump. Then, as described above, an operator can deliver a fluid F flush through the catheter lumen. In yet further embodiments, a catheter can have multiple lumens, such as a first lumen configured to receive a control element, and a second, separate, lumen configured to receive fluid from a fluid flush device.

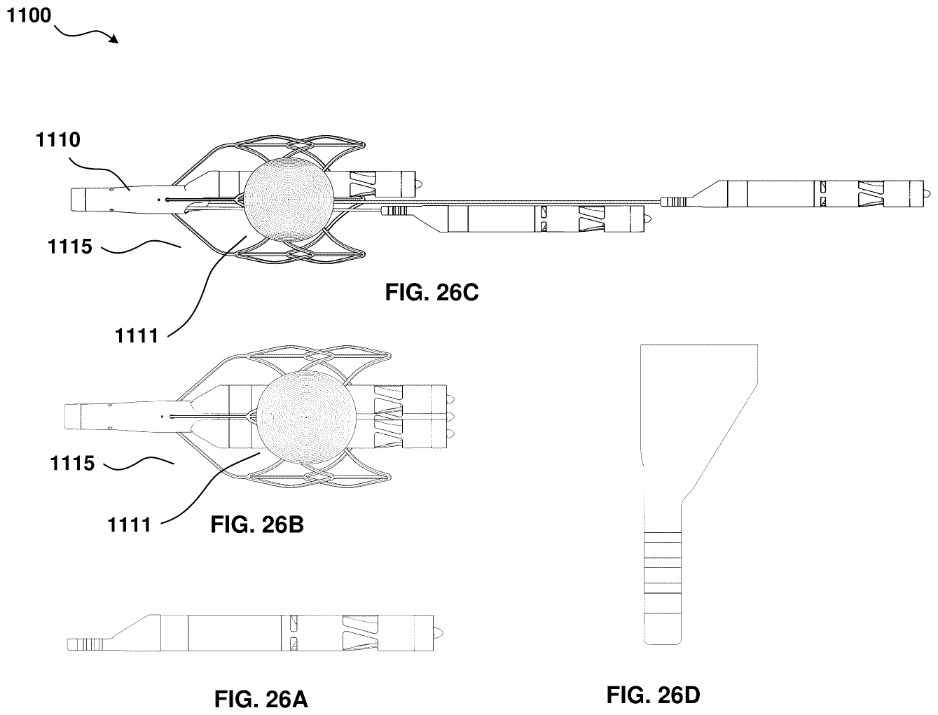

Consistent with the desire to minimize the footprint of the modular pump system in the subject's body, including minimizing any components (e.g., power transfer cables, wires, etc.) extending from the implanted pump and dock, through the subject's vasculature and/or outside the subject, in some embodiments, a modular pump system can transfer power wirelessly. For context, such wireless energy transfer may include features similar to those described in U.S. Pat. No. 10,143,788, Entitled “Transcutaneous Energy Transfer Systems,” hereby incorporated by reference in its entirety. FIGS. 26A-D illustrate such a modular pump system 1100, including a power source connector 1113 being or having a coil 1113c that is (ii) coupled to an anchor 1115 of a pump dock 1110, and (ii) configured to receive inductive power from a source located outside the subject's vasculature and/or body, according to an embodiment. Modular pump system 1100 can be similar to or the same as, in form and/or function, any of the modular pump systems described herein. Thus, portions of modular pump system 1100 are not described in further detail herein.

As shown in FIGS. 26B and 26C, pump dock 1110 includes an anchor 1115.

Coupled to and extending form an exterior surface of anchor 1115 is coil 111c (also referred to herein as “receiver coil”) formed of wire (and/or any other suitable component) into a disc shape. Modular pump system 1100 also includes an energy storage component (e.g., a battery) (not shown) operably coupled to coil 1111c and configured to store and/or transfer energy from coil 1111c to pumps 1130, 1140, 1150. In use, a transmitter (e.g., a transmitter coil) (not shown) can be brought into proximity of the implanted coil 111c to transfer power from the transmitter coil to receiver coil 111c, to in turn power the implanted components (pumps 1130, 1140, 1150) of modular pump system 1100. It should be noted that coil 1111c can accommodate expansion and compression of anchor 1115, e.g., during implantation and delivery, respectively.

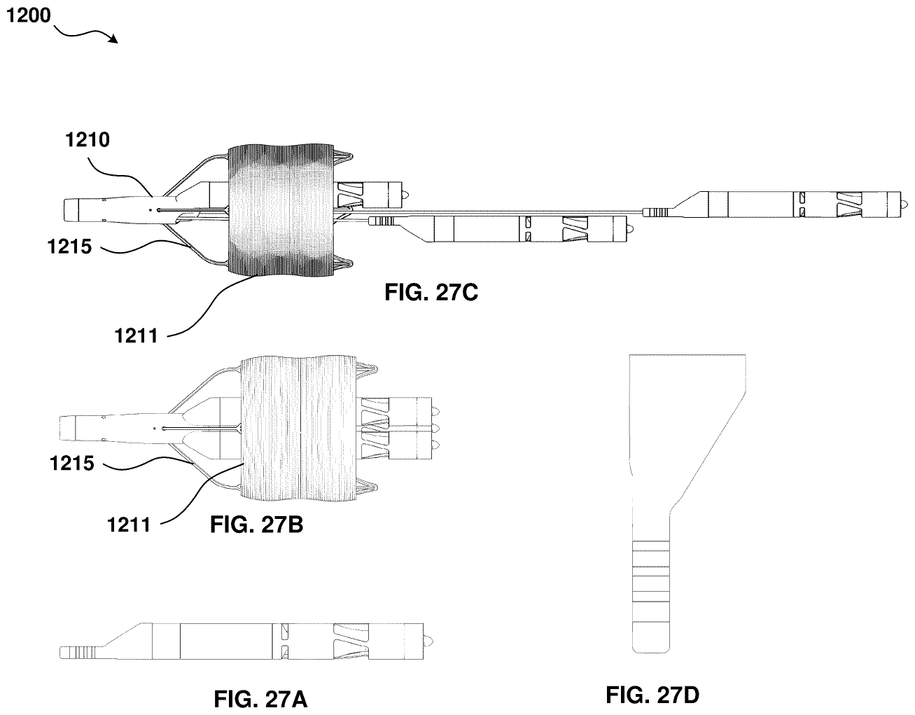

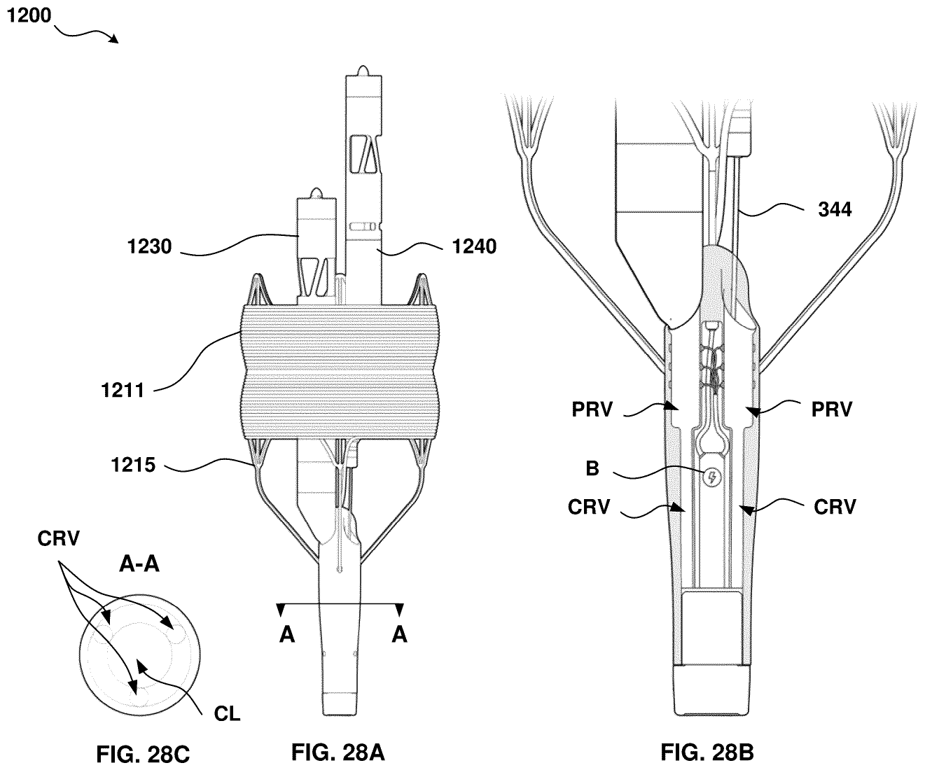

A receiver coil can have any suitable shape and size. FIGS. 27A-28C illustrate a modular pump system 1200 having a coil 1211c formed of wire (and/or any other suitable component) disposed circumferentially about anchor 1215, according to an embodiment. In this embodiment, e.g., relative to modular pump system 1100, coil 1211c can accommodate relatively more coils, or surface area. Further, although in this embodiment coil 1211c remains between the distal and proximal ends of anchor 1215, in some embodiments, coil 1211c may extend beyond the distal and/or proximal ends of anchor 1215. Also, in some embodiments, coil can have other shapes and/or configurations, e.g., it may have an oblique shape, and/or have wires more spaced apart, to accommodate expansion and compression of the anchor. Further, as shown best in FIG. 28B, pump dock 1210 includes a battery B that is electrically coupled to coil 1211.

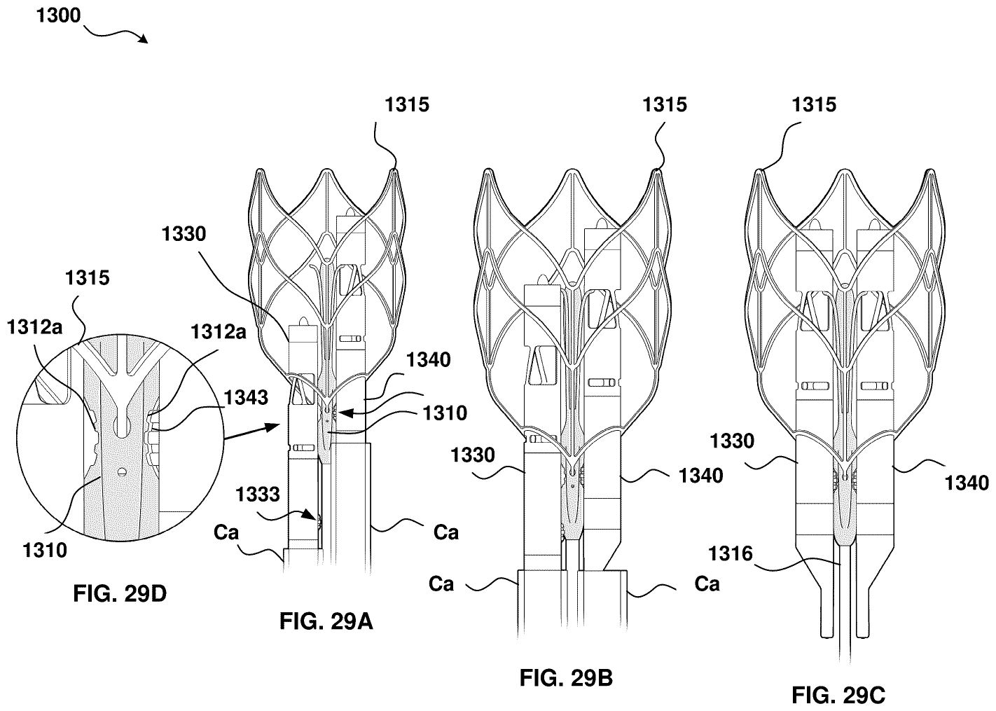

In some instances, it may be desirable to push each pump to deliver each pump through the subject's vasculature and to dock to the dock at the implantation site. FIGS. 29A-29D illustrate such a pump system 1300, according to an embodiment.

Modular pump system 1300 can be similar to or the same as, in form and/or function, any of the modular pump systems described herein. Thus, portions of modular pump system 1300 are not described in further detail herein.

For simplicity, the following description will focus on a single pump (i.e., pump 1330) and associated components; but it should be appreciated that such description can equally apply to the other pumps (i.e., pump 1340 and 1350) and associated components of modular pump system 1300.

In this embodiment, pump 1330 is configured to fit and be delivered to the implantation site within a lumen of catheter Ca. Although not shown in FIGS. 29A-29C, modular pump system 1300 includes control elements, each configured to be removably attached to a pump so that the control element may be released from the pump in use, similar to as described in several embodiments herein. In this embodiment, the control elements are sufficiently rigid such that an operator can push on a proximal end portion of the control elements to advance a distal end of the control elements (and the components coupled there, such as the pumps) within the subject's vasculature and such that each pump coupled to each control element can engage with and be docked to pump dock 1310. In some implementations, for example, each control elements can be a push rod. Alternatively, a relatively stiff cable or relatively stiff wire may be routed in the catheter control element of the pump to provide sufficient stiffness to the control element for pushing it. With control element (not shown) removably attached to pump 1330, for example, an operator can push on the control element and/or catheter Ca to advance pump 1330 (with catheter Ca) within the subject's vasculature dock pump 1330 with pump dock 1310 at the implantation site. In this embodiment, pump electrical connector 1333 (also referred to herein as device electrical connector) protrudes from an exterior surface of pump 1330 spaced from the prong of pump 1330, as shown, and is configured to correspondingly mate with dock electrical connector 1312a (also referred to herein as dock electrical connector) of pump dock 1310, which in this embodiment is a recess R configured to receive and be secured to pump electrical connector 1333, thereby providing both a physical securement function and an electrical connection to transfer energy from pump dock 1310 to pump 1330, similar to as described in connection with various embodiments herein. FIG. 29D illustrates a close-up detailed view of dock electrical connector 1312a, and pump electrical connector 1343 engaged with dock electrical connector 1312b.

Further, as shown across FIGS. 29A-29C, anchor 1315 defines cells through which the pumps (e.g., pump 1330 and 1340) can be advanced and disposed when docked to pump dock 1310.

Detailed embodiments of the present disclosure have been disclosed herein for purposes of describing and illustrating claimed structures and methods that can be embodied in various forms, and are not intended to be exhaustive in any way, or limited to the disclosed embodiments. Many modifications and variations will be apparent without departing from the scope of the disclosed embodiments. The terminology used herein was chosen to best explain the principles of the one or more embodiments, practical applications, or technical improvements over current technologies, or to enable understanding of the embodiments disclosed herein. As described, details of well-known features and techniques can be omitted to avoid unnecessarily obscuring the embodiments of the present disclosure.

While various embodiments have been described above, it should be appreciated that they have been presented by way of example only, and not limitation. Where schematics and/or embodiments described above indicate certain components arranged in certain orientations or positions, the arrangement of components may be modified. While the embodiments have been particularly shown and described, it will be appreciated that various changes in form and details may be made. The embodiments described herein can include various combinations and/or sub-combinations of the functions, components and/or features of the different embodiments described. Although various embodiments have been described as having particular features and/or combinations of components, other embodiments are possible having a combination (excluding mutually exclusive combinations) of any features and/or components from any of embodiments described herein.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be appreciated that the size, shape, and/or arrangement of the embodiments and/or components thereof can be adapted for a given use unless the context explicitly states otherwise.

References in the specification to “in some embodiments”, “one embodiment”, “an embodiment”, “an example embodiment”, and the like indicate that the embodiment(s) described can include one or more particular features, structures, or characteristics, but it shall be appreciated that such particular features, structures, or characteristics may or may not be common to each and every disclosed embodiment disclosed herein. Moreover, such phrases do not necessarily refer to any one particular embodiment per se. As such, when one or more particular features, structures, or characteristics is described in connection with an embodiment, it is to be understood that it is within the knowledge of those skilled in the art to affect such one or more features, structures, or characteristics in connection with other embodiments, where applicable, whether or not explicitly described.

Parameters, dimensions, materials, and configurations described herein are meant to be examples and that the actual parameters, dimensions, materials, and/or configurations will depend upon the specific application or applications for which the inventive teachings is/are used. It is, therefore, to be understood that the foregoing embodiments are presented by way of example only and that, within the scope of the appended claims and equivalents thereto, the embodiments can be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

Where methods and/or events described above indicate certain events and/or procedures occurring in certain order, the ordering of certain events and/or procedures may be modified. Additionally, certain events and/or procedures may be performed concurrently in a parallel process, when possible, as well as performed sequentially as described above.

As used herein, a component and/or a device can be, for example, any assembly and/or set of operatively-coupled electrical components associated with performing a specific function, and can include, for example, a memory, a processor, electrical traces, optical connectors, software (executing in hardware) and/or the like.

1.-63. (canceled)

64. A powered modular implantable medical device system, comprising:
a first implantable medical device that includes an electrical connector configured to receive power from a power source; and
a second implantable medical device that includes an electrical connector configured to intracorporeally connect the electrical connector of the first implantable medical device.

65. The system according to claim 64, wherein the electrical connector of the first implantable medical device and the electrical connector of the second implantable medical device are connectable together for intracorporeally operating the second implantable medical device through the first implantable medical device.

66. The system according to claim 64, wherein the first implantable medical device and the second implantable medical device are arrangeable in a slidable relationship relative to each other for intracorporeally connecting the electrical connector of the first implantable medical device and the electrical connector of the second implantable medical device together.

67. The system according to claim 64, wherein the first implantable medical device and the second implantable medical device are arrangeable in a slidable relationship relative to each other for intracorporeally disconnecting the electrical connector of the first implantable medical device and the electrical connector of the second implantable medical device from each other.

68. The system according to claim 64, wherein the second implantable medical device comprises a control element configured to establish an intracorporeal connection between the electrical connector of the first implantable medical device and the electrical connector of the second implantable medical device.

69. The system according to claim 68, wherein the control element is configured to be manipulable by an operator for establishing the intracorporeal connection.

70. The system according to claim 69, wherein the control element is configured to be extracorporeally manipulable by an operator for establishing the intracorporeal connection.

71. The system according to claim 68, wherein the control element is configured to be pullable relative to the first implantable medical device for establishing the intracorporeal connection.

72. The system according to claim 68, wherein the control element is further configured to disconnect the intracorporeal connection.

73. The system according to claim 72, wherein the control element is configured to be pushable relative to the first implantable medical device for disconnecting the intracorporeal connection.

74. The system according to claim 68, wherein the control element is configured to extend from the second implantable medical device.

75. The system according to claim 74, wherein the control element is configured to extend from the electrical connector of the second implantable medical device.

76. The system according to claim 68, wherein the control element is configured to be removably attachable to the second implantable medical device.

77. The system according to claim 68, wherein the control element is arrangeable along the first implantable medical device.

78. The system according to claim 64, further comprising a seal configured to sealingly engage a cavity of the electrical connector of the first implantable medical device.

79. The system according to claim 78, wherein the seal is further configured to receive the control element therethrough.

80. The system according to claim 79, wherein the seal is further configured to move inside the cavity upon connection of the electrical connector of the second implantable medical device to the electrical connector of the first implantable medical device.

81. The system according to claim 80, wherein the cavity defines a fluid flush port configured to evacuate a fluid from the cavity upon connection of the electrical connector of the second implantable medical device to the electrical connector of the first implantable medical device.

82. The system according to claim 68, wherein the first implantable medical device comprises a control element passage guide configured to slidably receive the control element therethrough.

83. The system according to claim 82, wherein the first implantable medical device further comprises a control component coupled to the control element passage guide, the control component being configured to at least partially slidably receive the control element therethrough.

84. The system according to claim 83, wherein the control component comprises an electrical conductor electrically coupled to the electrical connector of the first implantable medical device and electrically connectable to the power source.

85. The system according to claim 64, wherein the first implantable medical device and the second implantable medical device are transcatheterally implantable in vivo.

86. The system according to claim 85, wherein the first implantable medical device is a blood pump dock, and the second implantable medical device is a blood pump.

87. The system according to claim 64, wherein the electrical connector of the second implantable medical device is configured to intraluminally connect the electrical connector of the first implantable medical device.

88. The system according to claim 64, wherein the power source is a wireless power source, and the first implantable medical device is configured to wirelessly receive power from the wireless power source and to direct such power to the electrical connector of the first implantable medical device.

89. The system according to claim 64, further comprising an additional second implantable medical device that includes an electrical connector, wherein the first implantable medical device further includes another electrical connector configured to receive power from the power source, the electrical connector of the additional second implantable medical device being configured to intravascularly connect the other electrical connector of the first implantable medical device.

90. The system according to claim 89, wherein the other electrical connector of the first implantable medical device and the electrical connector of the additional second implantable medical device are connectable together for intravascularly operating the additional second implantable medical device through the first implantable medical device.

91. The system according to claim 90, wherein the first implantable medical device and the additional second implantable medical device are arrangeable in a slidable relationship relative to each other for intravascularly connecting the other electrical connector of the first implantable medical device and the electrical connector of the additional second implantable medical device together, and for intracorporeally disconnecting the other electrical connector of the first implantable medical device and the electrical connector of the additional second implantable medical device from each other.

92. The system according to claim 91, wherein the additional second implantable medical device comprises a control element configured to establish an intracorporeal connection between the other electrical connector of the first implantable medical device and the electrical connector of the additional second implantable medical device.

93. The system according to claim 92, wherein the control element of the additional second implantable medical device is configured to be extracorporeally pullable, relative to the first implantable medical device, by an operator for intravascularly connecting the other electrical connector of the first implantable medical device and the electrical connector of the additional second implantable medical device together, and is further configured to be extracorporeally pushable, relative to the first implantable medical device, by the operator for intravascularly disconnecting the other electrical connector of the first implantable medical device and the electrical connector of the additional second implantable medical device from each other.

94. The system according to claim 93, wherein each of the second implantable medical device and the additional second implantable medical device is dockable to and undockable from the first implantable medical device.

95. The system according to claim 94, wherein the first implantable medical device is a transcatheter blood pump dock, and each of the second implantable medical device and the additional second implantable medical device is a respective transcatheter blood pump.

96. A method of implanting a powered modular implantable medical device system to an intracorporeal implantation site of a subject, the method comprising:
delivering the powered modular implantable medical device system to the intracorporeal implantation site; the powered modular implantable medical device system comprising a first implantable medical device having an electrical connector, and a second implantable medical device having an electrical connector configured to intracorporeally connect the electrical connector of the first implantable medical device; and
intraluminally connecting the electrical connector of the first implantable medical device and the electrical connector of the second implantable medical device together.

97. A method of explanting a powered modular implantable medical device system from an intracorporeal implantation site of a subject, the powered modular implantable medical device system comprising a first implantable medical device having an electrical connector, and a second implantable medical device having an electrical connector intracorporeally connected to the electrical connector of the first implantable medical device, the method comprising:
intraluminally disconnecting the electrical connector of the first implantable medical device and the electrical connector of the second implantable medical device from each other; and
retrieving the first implantable medical device and the second implantable medical device from the intracorporeal implantation site.
